(19) United States
(12) Patent Application Publication
(10) Pub. No.: US 2024/0117436 A1
(43) Pub. Date: Apr. 11, 2024

(54) METHODS OF TREATING LIPEDEMA INCLUDING AKR1C2 AS A THERAPEUTIC TARGET

(71) Applicant: MAGI EUREGIO SCS, Bolzano (IT)

(72) Inventors: Matteo BERTELLI, San Felice del Benaco (IT); Elena MANARA, Dro (IT); Paolo Enrico MALTESE, Rovereto (IT); Sandro MICHELINI, Roma (IT); Serena MICHELINI, Roma (IT); Maria Rachele CECCARINI, Marsciano (IT); Pietro CHIURAZZI, Roma (IT); Karen Louise HERBST, Tucson, AZ (US); Jurgen KAFTALLI, Bolzano (IT); Kevin DONATO, Bolzano (IT); Andrea BERNINI, Siena (IT)

(73) Assignee: MAGI EUREGIO SCS, Bolzano (IT)

(21) Appl. No.: 18/516,241

(22) Filed: Nov. 21, 2023

Related U.S. Application Data
(63) Continuation-in-part of application No. 17/734,708, filed on May 2, 2022.
(60) Provisional application No. 63/183,313, filed on May 3, 2021.

(51) Int. Cl. C12Q 1/6883; A61K 31/5517; G01N 33/74

(52) U.S. Cl. CPC C12Q 1/6883; A61K 31/5517; G01N 33/743; C12Q 2600/106; C12Q 2600/136; C12Q 2600/156; C12Q 2600/158; G01N 2800/52

(57) ABSTRACT

The present invention identifies further to AKR1C1 also AKR1C2 as a lipedema-associated gene. The invention provides methods for diagnosing or assessing an individual's susceptibility to lipedema by the analysis of the AKR1C genes, in particular of the AKR1C2 gene, or the expression levels of its product and related metabolites. Also provided are therapeutic methods for treating a patient or methods for prophylactically treating an individual susceptible to lipedema.

### CROSS-REFERENCE TO RELATED APPLICATIONS

This application is a Continuation-in-Part of application Ser. No. 17/734,708, filed on May 2, 2022, which claims priority under 35 U.S.C. § 119(e) to U.S. Provisional Application No. 63/183,313, filed on May 3, 2021, all of which are hereby expressly incorporated by reference into the present application.

### REFERENCE TO SEQUENCE LISTING SUBMITTED VIA EFS-WEB

The application contains a Sequence Listing which has been submitted electronically in .XML format and is hereby incorporated by reference in its entirety. Said .XML copy, created on Nov. 17, 2023, is named “MAGI CIP.xml” and is 80,206 bytes in size. The sequence listing contained in this .XML file is part of the specification and is hereby incorporated by reference herein in its entirety.

### FIELD OF THE INVENTION

This invention relates to methods to prevent and treat human lipedema and to methods for diagnosis of lipedema or the determination of a predisposition for lipedema.

### BACKGROUND

Lipedema is a chronic and progressive pathologic condition mainly characterized by an abnormal body fat distribution. It affects extremities with abnormal fat deposition in thighs and legs and in some cases also the arms, while the trunk, hands and feet remain unaffected [Kruppa et al., 2020]. Lipedema is an autosomal dominant genetic disease that mainly affects women. It is characterized by excess deposition of subcutaneous adipose tissue, pain, and anxiety [Paolacci et al., 2019; Precone et al., 2019; Michelini et al., 2022]. The genetic and environmental etiology of lipedema is still largely unknown, and while rare, it is suggested to be misdiagnosed as obesity or lymphedema [Warren et al., 2007; Forner-Codero, 2012; Fife et al., 2010]. Lipedema patients can be distinguished from these two conditions by a series of features such as body disproportion, bilateral symmetry, hematoma tendency and scarce influence of diet, exercise and bariatric surgery.

Contrary to obese patients, the increase of fat in lipedema often causes symptoms such as pain and increased vascular fragility and is not responsive to diet or exercise. Contrary to lymphedema, the tissue in lipedema is soft to the touch. Involvement of steroid hormones was postulated. Indeed, its manifestations commonly arise in females in phases of hormonal changes, and steroid hormones are known to be involved in adipogenesis, anxiety, and pain, three common features of lipedema.

It has been estimated that about 10% of the woman are affected by lipedema worldwide [Buck D W and Herbst K L, 2016]. Male cases have been described in very few reports. For this reason, the involvement of sexual hormones in the etiology of the disease has been postulated several times [Torre et al., 2018; Bauer et al., 2019]. In line with this hypothesis, manifestations commonly arise in phases of hormonal changes (puberty, pregnancy or menopause) in females [Torre et al., 2018]. There is very strong evidence of a genetic base for the condition, since an autosomal dominant hereditary pattern was found in many families [Buso et al., 2019].

While genetic factors apparently regulate subcutaneous adipose tissue distribution, so far, no monogenic cause of non-syndromic primary lipedema have been discovered until the inventors' work.

Generally, patients with lipedema undergo differential diagnosis from other disorders, and other genes are screened to exclude the patient as having a known diagnosis of another disorder of subcutaneous adipose tissue (ADRA2A, AKT2, ALDH18A1, CIDEC, LIPE, LMNA, MFN2, NSD1, PALB2, PLIN1, POU1F1, PPARG, TBL1XR1) and of localized lipodystrophies (AGPAT2, AKT2, BSCL2, CAV1, CAVIN1 (PTRF), CIDEC, LIPE, LMNA, PLIN1, PPARG, ZMPSTE24).

Therapies are performed to help relieve symptoms and prevent frustration. When possible, a conservative management is suggested and this includes manual lymph drainage, appropriate compression therapy with custom-made, flat-knitted compressive clothing, psychosocial therapy, patient education on self-management, physiotherapy and exercise therapy (such as low impact, cycling, walking or other exercise or movements), dietary counseling and weight management.

Lipedema fat is resistant to diet therapy. Current dietary approaches are aimed at lowering body weight through a hypocaloric diet, inhibiting systemic inflammation with antioxidant and anti-inflammatory components and reducing water retention [Di Renzo et al., 2021].

In some cases, if symptoms impair quality of life, the potential indication for surgery should be evaluated. Liposuction therapeutic benefit has not yet been evaluated in any randomized, controlled trials. Liposuction can reduce leg circumference, pain, feeling of tightness, tendency to form hematomas, improving quality of life. In highly advanced stages of the disease (i.e. in presence of lymphedema and fibrosis) dermato-fibro-lipectomy may be indicated.

### SUMMARY OF THE INVENTION

This invention provides methods for diagnosing lipedema or identifying agents for treating a patient having lipedema or a predisposition for lipedema. The methods comprise one or more of the following steps:
detecting step to identify variants in the sequence of AKR1C1 or AKR1C2 gene from gDNA (genomic DNA). Single nucleotide polymorphism (SNP) analysis is also useful for detecting differences between alleles of AKR1C1 or AKR1C2 genes, that reside within a region of human chromosome 10. Within this region, a great number of known SNPs have been reported to date;
detecting step comprises quantifying mRNA encoding an AKR1C1 or AKR1C2 isoform in a biological sample (blood, urine and adipose tissue specimens);
detecting increment or reduction of AKR1C1 or AKR1C2 enzymatic substrate or product (i.e. steroid derivatives and prostaglandins) in a biological sample (blood, urine and adipose tissue specimens) in a lipedema patient compared to controls. The biological sample can be screened with an antibody that specifically binds to AKR1C1 or AKR1C2 enzymatic substrate or product or the biological sample can be treated or converted by AKR1C1 or AKR1C2 enzyme;
identifying natural and synthetic molecules capable of modulating AKR1C1 or AKR1C2 with possible therapeutic effect on lipedema.
   This invention further proposes treatments of lipedema, in particular drug or nutraceutical treatments of lipedema.

Only the identification of AKR1C1 and AKR1C2 as the first lipedema-associated genes rendered the diagnostic and therapeutic approaches herein described possible. The identification of the gene and its linkage to lipedema opened the way to diagnose and treat the disease of lipedema. Since AKR1C1 and AKR1C2 are the first genes associated with the molecular diagnosis of non-syndromic lipedema, there are currently no molecular diagnostic alternatives.

Indeed, with their study, the inventors argue in favor of the involvement of AKR1C1 and AKR1C2 in lipedema [Michelini et al., 2020; reference herein]. AKR1C1 is a gene highly expressed in the subcutaneous tissue and it has been suggested that its activity in the regulation of steroid hormone levels plays an important role in the accumulation of subcutaneous fat depots. The enzyme expressed by this gene, the 20α-hydroxysteroid dehydrogenase (20α-HSD), metabolizes progesterone, a hormone that prompts lipogenesis [Blanchette et al., 2005]. Up to the inventors' previous and current findings, AKR1C1 or AKR1C2 have not been implicated in any genetic condition characterized by or including lipedema among its clinical manifestations.

The association may be due to rare genetic variants or common polymorphisms that alter enzymatic function and can also be caused by epigenetic alterations.

In fact, the inventors previously found that AKR1C1, an essential enzyme for steroid hormone regulation, is mutated in a family affected by lipedema [Michelini et al., 2020], suggesting that these hormones may play a role in the pathogenesis of the disease. It is known that sex hormones also determine the anatomical site of the accumulation of adipose tissue, and dysfunction of sex steroids result in abnormal fat distribution in predisposed subjects, especially in females at the time of puberty [Grigoriadis et al., 2021; Gavin et al., 2013]. The homeostasis of steroid hormones is finely regulated by enzymes such as aldo-keto reductases (ARK1C), hydroxysteroid dehydrogenase (HSD) and aromatases expressed in adipocytes, preadipocytes, and mature adipose tissue [Tchernof et al., 2015; Blouin et al., 2009].

The four human AKR1Cs are multifunctional enzymes with overlapping activities on a broad range of substrates. They possess approximately 320 amino acid residues and share at least 84% amino acid sequence identity. AKR1C1 and 1C2 in particular differ by only seven amino acids, with only one amino acid difference at the active site [see description below]. All four AKR1Cs can exert 3-, 17- and 20-ketoreductase activity, though each has its own distinct preferences for position, stereochemistry and substrate. AKR1C1 is the major 20α-reductase that inactivates progesterone, whereas AKR1C2 preferentially acts as a 3α-reductase, with particular importance in the deactivation of dihydrotestosterone (DHT) to 3α-Adiol [Penning et al., 2019]. Progesterone and DHT play opposite roles with regard to fat accumulation, with progesterone prompting lipogenesis and DHT inhibiting adipogenesis [Kiani et al., 2021].

To support the claim that AKR1C2 overexpression may be a feature of lipedema, the inventors described the AKR1C1 L54V variant found in one of the studied lipedema patients, which turns AKR1C1 into an enzyme possessing AKR1C2 activity [Zhang et al., 2014], as well as the AKR1C2 Ser320PheTer2 variant causing a deletion in the C-term tail of the protein. The inventors then decided to study the expression levels of AKR1C2 in a separate cohort of lipedema patients, and indeed, alteration in AKR1C2 mRNA expression was detected.

Finally, the inventors observed that variants gathered from the GWAS catalog database that were typical to obesity patients, lied in AKR1C2 promoter regions (known to affect fat deposition). Considering that lipedema is often misdiagnosed as obesity, these variants found in AKR1C2 promoters provided new insights into the correlation of AKR1C genes to lipedema.

The role of leucine 54 in substrate selectivity has been already elucidated [Penning et al., 2019; Couture et al., 2003]. Its bulky side chain significantly confines the spatial movement of the steroid in its cavity, restricting the flexibility provided by valine 54 in AKR1C2 [Zhang et al., 2014; Couture et al., 2003]. Of the seven amino acids that are different between AKR1C1 and AKR1C2, one is located in the active site at position 54. The replacement of leucine 54 in AKR1C1 with valine 54 found in AKR1C2, is the only mutation that generated an enzyme with identical properties to AKR1C2, further indicating the importance of residue 54 in determining the substrate specificity of the two enzymes [Matsuura et al., 1997] while the reverse mutation V54L in AKR1C2 converts the enzyme into AKR1C1 (FIG. 5: shows respective activities of AKR1C1 and AKR1C2, with the L54V variant turning AKR1C1 activity into that of AKR1C2), enhancing the 20α-HSD activity and significantly reducing the 3α-HSD3 activity [Zhang et al., 2014].

The AKR1C enzymes exert their HSD activity mainly in subcutaneous adipose tissue as the reduction and inactivation of steroid hormones [Penning, Trevor M., 1997; Blouin et al., 2006]. AKR1C1, which is highly expressed in adipocytes and subcutaneous fat, is responsible for inactivating progesterone to 20α-hydroxyprogesterone, a main metabolite in isolated mature adipocytes. In this way, AKR1C1 decreases the levels of progesterone in peripheral adipose tissue [Brozic et al., 2009; Michelini et al., 2020]. AKR1C1 also reduces DHT to its 3β metabolite, 5α-androstane-3β,17β-diol (3β-Adiol), which is a potent agonist of the estrogen receptor beta (ERβ). ERβ may enhance the lipid burning process in adipose tissue [Katzer et al., 2021], suggesting that loss of this function of AKR1C1 may further contribute to fat deposition. In rats, progesterone reverses the weight-reducing actions of estradiol [Gray et al., 1981; Wade, G N, 1975]. This suggests that a diminished activity of AKR1C1 could lead to high levels of progesterone in adipocytes and a consequent increase of lipogenesis mediated by this hormone [Michelini et al., 2020]. Meanwhile, AKR1C2 is important for DHT inactivation in preadipocytes, and overexpression of it might lead to the elimination of the androgen inhibitory effects on adipogenesis [Penning et al., 2019]. Therefore, this single mutation is involved in lipedema by affecting the steroid homeostasis two-fold: diminishing the activity of AKR1C1 on the reduction of progesterone which mediates lipogenesis, while enhancing the activity of AKR1C2 on the reduction and inactivation of DHT which inhibits adipogenesis.

As previously described, in one method for the diagnosis of lipedema and/or for the individuation of treatments thereof, the variants of step (i) are detected from gDNA, in particular by single nucleotide polymorphism (SNP) analysis for detecting differences between alleles of AKR1C1 genes, that reside within a region of human chromosome 10, or detected through NGS (Next Generation Sequencing) or Sanger technologies.

In an advantageous embodiment of the method for the diagnosis of lipedema and/or for the individuation of treatments thereof, the variants of step (i) are selected from known loss-of-function (LoF) SNPs indicated in table 1 or from a list of selected SNPs as indicated in table 2 or 4. The SNPs can, for example, be selected on the basis of the following criteria: only missense variants; absent in homozygous state; frequency below 0.1%. The selected variants are subsequently preferably studied by functional modelling to verify their impact, for example in terms of binding affinity to certain compounds. This permits to study for one or more particular variants found in a patient the binding affinity to pharmaceutically active compounds, to find the compound that best fits for the particular variant and thus for the patient being affected by this variant.

Preferably, the AKR1C1 variants are selected from the group consisting of: c.840C>A (p.Asn280Lys), or are selected from c.160T>G (p.Leu54Val), c.162A>T (p.Leu54Phe), c.638T>A (p.Leu213Gln), the p.Leu54 and p. Leu213 variants being particularly preferred. The four variants above are particularly interesting as they have been found in lipedema patients.

In particular, the missense variant p.(Leu213Gln) in AKR1C1, the gene encoding for an aldo-keto reductase catalyzing the reduction of progesterone to its inactive form, 20-α-hydroxyprogesterone, suggests a partial loss-of-function resulting in a slower and less efficient reduction of progesterone to hydroxyprogesterone and an increased subcutaneous fat deposition in variant carriers. The p.(Leu213Gln) variant, to the knowledge of the inventors, is the first one ever identified in a lipedemia family.

Being an inducible gene [Pallai et al., 2010], AKR1C1 expression in the blood can be a marker of the disease. Similarly, urinary and blood plasma or serum metabolites can be used as disease markers and have diagnostic value.

In another embodiment of the method for the diagnosis of lipedema and/or for the individuation of treatments thereof, the mRNA of step (ii) or the enzymatic substrate or product or metabolite of step (iii) is detected in a biological sample, in particular in blood, urine and/or adipose tissue specimens.

In a preferred embodiment, the enzymatic substrate or product or metabolite of step (iii) is a steroid derivative or a prostaglandin.

Preferably, the biological sample of step (iii) is screened with an antibody that specifically binds to the AKR1C1 enzymatic substrate or product or metabolite or the biological sample is treated or converted by AKR1C1 enzyme.

Preferably, the enzymatic substrate or product in step (iii) is selected among 20α-hydroxysteroid dehydrogenase (20α-HSD); PGF2α and its derivatives, in particular by measurement of 15-keto-13,14-dihydro-PGF2α, the major metabolite of PGF2α in plasma; or isoprostane 8-iso-Prostaglandin F2α (8-iso-PGF2α).

In a preferred embodiment of the method for the diagnosis of lipedema, in step (iii) the levels of at least one of the following metabolites 3α-Hydroxy-5α-pregnan-20-one, 3α-Hydroxy-5β-pregnan-20-one, 3β-Hydroxy-5α-pregnan-20-one, 3β-Hydroxy-5β-pregnan-20-one, 5α-Pregnane-3,20-dione, 5β-Pregnane-3,20-dione, Pregn-4-ene-3,20-dione, 20α-Hydroxy-pregn-4-ene-3-one, 5α-Pregnane-3α,20α-diol, 5β-Pregnane-3α,20α-diol, 5α-Androstan-17β-ol-3-one, 5α-androstane-3α,17β-diol, 21-hydroxy-5α-pregnan-20-one, 3α,21-dihydroxy-5α-pregnan-20-one, Pregnanetriol/17-hydroxypregnanol one, 15-keto-13,14-dihydro-PGF2α, in particular 8-iso-Prostaglandin F2α progesterone and/or 5alpha-dihydrotestosterone is determined in a body fluid.

In another embodiment of the method for the diagnosis of lipedema, in step (iii) the ratio (androstanediol^{1.5}×20β-DH-cortisone)/(20β-DH-cortisone+[cortisol×log(estriol)] in a body fluid is determined.

AKR1C1 is a target of natural and synthetic molecules capable of modulating its activity. Benzodiazepines such as medazepam represent a class of non-competitive inhibitors of AKR1C1. Synthetic derivatives of pyrimidine, phthalimide and anthranilic acid potently inhibited AKR1C1 (Brozic et al., 2009). Compounds provided with a core structure of steroid carboxylate and flavones are instead AKR1C1 competitive inhibitors. Among natural compounds, liquiritin has been discovered as a selective and potent AKR1C1 inhibitor capable of reducing the progesterone metabolism in cells [Zeng et al., 2019].

Prostanoids, acting via peroxisome proliferator-activated receptor gamma (PPARγ), a fundamental receptor in fatty acid storage and glucose homeostasis, have been proposed as potent regulators of fat cell differentiation. Indeed, in vitro studies showed that prostaglandin J2 (PGJ2) binds and activates PPARγ acting as a potent adipogenic hormone; inversely, prostaglandin F(2α) (PGF2α), which has PPARγ antagonist properties, is a potent antiadipogenic factor [Quinkler et al., 2006; Volat et al., 2012]. Another proof of the involvement of prostaglandins (PG) in the regulation of adipocyte differentiation came from the use of PG analogues as hypotensive agents in the treatment of glaucoma, extensively described in literature reports. Indeed, patients treated with topical therapies based on PG analogues showed periorbital fat changes as an adverse effect. These molecules can directly lead to reduced orbital fat by inhibiting adipogenesis [Taketani et al., 2014]. Aldo-keto reductases have been reported as major regulators of white adipose tissue development with antiadipogenic properties supported by PGF2α synthase activity [Quinkler et al., 2006; Volat et al., 2012]. Indeed, PGF2α can be synthesized from PGD2 and PGE2 by the enzymes AKR1C (1, 2 and 3) [Quinkler et al., 2006; Dozier et al., 2008] and Akr1b7 [Volat et al., 2012]. In vitro studies demonstrated that PGD2 enhances adipocyte differentiation while PGE2 and PGF2α suppress adipogenesis [Miller et al., 1996].

A further aspect refers to a method of treating and/or preventing of human lipedema in a subject, the method comprising administering or applying to a subject in need thereof a therapeutically effective amount of a compound of natural or synthetic origin, preferably contained in a food supplement, cream or ointment, suitable for modulating the activity of AKR1C1 or of prostaglandins.

In one embodiment of the method of treating and/or preventing of human lipedema in a subject, the compound modulates the catalytic activity of the AKR1C1 enzyme, and comprises at least one of the compounds indicated in table 6, in particular benzodiazepines, such as medazepam, derivatives of pyrimidine, phthalimide and anthranilic acid, competitive inhibitors with a core structure of steroid carboxylate and flavones, and liquiritin. Advantageously, the compound is selected from the group consisting of flavanone, flavone, 3-hydroxyflavone, 5-hydroxyflavone, equilin, diazepam, 20α-hydroxydydrogesterone, coumarin, glycyrrhetinic acid, 7-hydroxyflavone and 3,7-dihydroxyflavone.

In another embodiment of the method of treating and/or preventing of human lipedema in a subject, the compound is suitable for modulating prostaglandins and comprises at least one of the compounds indicated in table 7.

Variants of the method of treating and/or preventing of human lipedema in a subject foresee, that the step of administering or applying to a subject in need thereof a therapeutically effective amount of a compound of natural or synthetic origin is preceded by a step for the diagnosis of lipedema according to the invention that confirmed the tested person is affected by lipedema. Advantageously, the confirmation of the fact that the tested person is affected by lipedema is obtained by the detection of a biomarker in a body fluid in a concentration exceeding a determined limit value.

A further aspect relates to a composition for the treatment of human lipedema, in particular in the form of a food supplement, cream or ointment, comprising a compound that modulates the catalytic activity of the AKR1C1 enzyme or of prostaglandins, in particular at least one of the components indicated in tables 6-10.

An additional aspect of the invention relates to a food supplement comprising the composition according to the invention. Another aspect of the invention relates to a cream comprising the composition according to the invention. A final aspect of the invention refers to an ointment comprising the composition according to the invention.

### BRIEF DESCRIPTION OF THE DRAWINGS

FIG. 1 depicts in a molecular simulation the structure of AKR1C1.

FIG. 2 is a plot relating single amino acids of AKR1C1 to their progesterone binding energy.

FIG. 3 depicts a detail of the structure of AKR1C1 and binding situations to progesterone.

FIG. 4 is a plot relating single amino acids of AKR1C1 to their NAPD(H) binding energy.

FIG. 5 illustrates schematically the role of Leu54 in substrate activity for AKR1C1 and AKR1C2.

FIG. 6 depicts in a molecular simulation the disrupted interaction between the steroid progesterone and the AKR1C1 enzyme due to the replacement of the same Leu54 by phenylalanine.

FIG. 7 compares in a molecular simulation the cofactor binding contribution of Asn280 and Gln279.

FIG. 8 depicts the L213Q family tree of the inventors' previous study [Michelini et al., 2020].

FIG. 9 shows the relative expression of AKR1 C1 and AKR1C3 in different groups of the study of FIG. 8.

FIG. 10 shows the conformation of 5α-DHT into the binding pocket of AKR1C2.

FIG. 11 depicts the distance between C3 of 5α-DHT and the hydroxyl group of Tyr55 and

the distance between C3 of 5α-DHT and C4N of NADPH.

### DETAILED DESCRIPTION OF INVENTION

Diagnostic methods can comprise the sequencing (through next generation sequencing [NGS] or Sanger technologies) of the AKR1C1 gene, or portions of it, or through whole genome and whole exome approaches for the diagnosis of lipedema. Single nucleotide polymorphism (SNP) analysis is also useful for detecting differences between alleles of AKR1C1 genes that reside within a region of human chromosome 10. Within this region, about 700 known SNPs have been reported to date. A list of known loss-of-function (LoF) SNPs is shown in table 1. In addition, a series of SNPs to have effect on protein function and an association with lipedema selected on the basis of the following criteria are listed in table 2: only missense variants; absent in homozygous state; frequency below 0.1%.

TABLE 1
AKR1C1 known LoF variants

| Transcript | Protein | | | Allele |
|---|---|---|---|---|
| Consequence | Consequence | rsID | VEP Annotation | Frequency % |
| c.84 + 1G > T | | rs748912524 | splice_donor_variant | 0.00039896 |
| c.64C > T | p.Gln22* | rs1430171919 | stop_gained | 0.000475064 |
| c.90 + 2T > G | | rs568245058 | splice_donor_variant | 0.029441491 |
| c.100delG | p.Ala34Leufs*2 | rs763666450 | frameshift_variant | 0.000399131 |
| c.134delG | p.Gly45Alafs*30 | rs1138573 | frameshift_variant | 0.000397874 |
| c.172G > T | p.Glu58* | rs1302342979 | stop_gained | 0.000397772 |
| c.81 − 1G > T | | rs530323152 | splice_acceptor_variant | 0.000397779 |
| c.81 − 1G > A | | rs530323152 | splice_acceptor_variant | 0.003580009 |
| c.81 − 1G > C | | rs530323152 | splice_acceptor_variant | 0.000397779 |
| c.196C > T | p.Arg66* | rs201114964 | stop_gained | 0.013793201 |
| c.252 + 2T > C | | rs775284743 | splice_donor_variant | 0.00122379 |
| c.258G > A | p.Trp86* | rs143557246 | stop_gained | 0.000801366 |
| c.271C > T | p.Arg91* | rs139089923 | stop_gained | 0.001775833 |
| c.286C > T | p.Arg96* | rs143132605 | stop_gained | 0.019841832 |
| c.369 + 2T > C | | rs777080970 | splice_donor_variant | 0.008869274 |
| c.394delG | p.Asp132Metfs*44 | rs1188750311 | frameshift_variant | 0.000600478 |
| c.394_397dupGATG | p.Glu133Glyfs*2 | rs1188750311 | frameshift_variant | 0.000600478 |
| c.403G > T | p.Gly135* | rs763837541 | stop_gained | 0.000574132 |
| c.448 − 1G > A | | | splice_acceptor_variant | 0.000397766 |
| c.514C > T | p.Gln172* | rs1220725793 | stop_gained | 0.000397627 |
| c.570 + 1G > A | | rs770791176 | splice_donor_variant | 0.000795494 |
| c.615G > A | p.Trp205* | rs1272520735 | stop_gained | 0.000416171 |
| c.649dupA | p.Ser217Lysfs*58 | rs370014498 | frameshift_variant | 0.000397864 |
| c.667C > T | p.Arg223* | rs781923069 | stop_gained | 0.000796768 |
| c.680 + 1G > A | | rs142084692 | splice_donor_variant | 0.00850732 |
| c.680 + 1G > C | | rs142084692 | splice_donor_variant | 0.003899188 |
| c.680 + 2T > C | | rs757191838 | splice_donor_variant | 0.00079734 |
| c.681 − 1G > A | | rs782472454 | splice_acceptor_variant | 0.000400352 |
| c.681G > A | p.Trp227* | rs782615031 | stop_gained | 0.002134426 |
| c.698delC | p.Pro233Argfs*22 | rs781955346 | frameshift_variant | 0.000712728 |
| c.741delG | p.Lys247Asnfs*8 | rs781870854 | frameshift_variant | 0.001989036 |
| c.748C > T | p.Arg250* | rs782207877 | stop_gained | 0.001988894 |
| c.846 + 1G > A | | rs782167092 | splice_donor_variant | 0.000545756 |
| c.846 + 1G > T | | rs782167092 | splice_donor_variant | 0.003820293 |
| c.910C > T | p.Arg304* | | stop_gained | 0.005656535 |
| c.929 + 1G > A | | rs781944824 | splice_donor_variant | 0.00209389 |
| c.945delT | p.Asn316Ilefs*15 | rs782460823 | frameshift_variant | 0.001235799 |
| c.962delA | p.Asp321Valfs*10 | | frameshift_variant | 0.000818391 |
| c.969T > G | p.Tyr323* | rs201500205 | stop_gained | 0.059779068 |

TABLE 2
AKR1C1 selected variants

| Transcript | | | | |
|---|---|---|---|---|
| Consequence | | | | |
| AKR1C1: | Protein | | | Allele |
| NM 001353.6: | Consequence | rsID | VEP Annotation | Frequency % |
| c.160T + G¹ | p.Leu54Val | rs138675307 | missense_variant | 0.080147155 |
| c.162A + T¹ | p.Leu54Phe | rs14929564 | missense_variant | 0.080147155 |
| c.911G > T² | p.(Arg304Leu) | — | missense_variant | |
| c.381A + T² | p.(Glu127Asp) | — | missense_variant | |
| c.664_665delCAinsAT² | p.(His222Ile) | — | missense_variant | |
| c.664_665delCAinsTC² | p.(His222Ser) | — | missense_variant | |
| c.919_920delACinsGT² | p.(Thr307Val) | — | missense_variant | |
| c.925_926delGAinsCT | p.(Asp309Leu) | — | missense_variant | |
| (p.Asp309Leu)² | | | | |
| c.914A > T² | p.(Tyr305Phe) | — | missense_variant | |
| c.638T > A³ | p.Leu213Gln | rs372782197 | missense_variant | 0.011188627 |
| c.22G > C | p.Val8Leu | rs752938448 | missense_variant | 0.000397735 |
| c.22G > T | p.Val8Leu | rs752938448 | missense_variant | 0.000397735 |
| c.32A > G | p.Asn11Ser | rs1446558895 | missense_variant | 0.000397772 |
| c.5G > A* | p.Gly2Glu | rs1405103238 | missense_variant | 0.000475638 |
| c.82A > G* | p.Met28Val | rs1187727403 | missense_variant | 0.003225598 |
| c.97A > G | p.Lys33Glu | rs1177376359 | missense_variant | 0.000399109 |
| c.104T > C | p.Leu35Ser | rs1174379434 | missense_variant | 0.000397988 |
| c.139C > A | p.Arg47Ser | rs748193660 | missense_variant | 0.000397791 |
| c.163T > C | p.Tyr55His | rs1564314801 | missense_variant | 0.000397725 |
| c.168T > A | p.Asn56Lys | | missense_variant | 0.000397747 |
| c.184G > A | p.Gly62Arg | rs1274415938 | missense_variant | 0.000397829 |
| c.272G > T | p.Arg91Leu | rs375752583 | missense_variant | 0.000399304 |
| c.274C > A | p.Pro92Thr | rs763383627 | missense_variant | 0.000399081 |
| c.290C > G | p.Pro97Arg | rs756379873 | missense_variant | 0.000398594 |
| c.298G > C | p.Glu100Gln | rs1564315232 | missense_variant | 0.000398318 |
| c.338T > C | p.Leu113Pro | rs1344076147 | missense_variant | 0.000398362 |
| c.355C > A | p.Pro119Thr | rs752532298 | missense_variant | 0.000398314 |
| c.392A > T | p.Cys131Ile | rs369662093 | missense_variant | 0.000602736 |
| c.394G > T | p.Asp132Tyr | rs1364894460 | missense_variant | 0.000601214 |
| c.566A > G | p.Asn189Ser | rs771829414 | missense_variant | 0.00039769 |
| c.584A > G* | p.Asp195Gly | rs1407820595 | missense_variant | 0.000417011 |
| c.607C > A* | p.Pro203Thr | rs962503713 | missense_variant | 0.000415866 |
| c.607C > G* | p.Pro203Ala | rs962503713 | missense_variant | 0.000415866 |
| c.575A > C | p.Glu192Ala | rs1564317029 | missense_variant | 0.000399683 |
| c.616T > G | p.Cys206Gly | rs782505662 | missense_variant | 0.00039807 |
| c.698C > T | p.Pro233Leu | rs370027719 | missense_variant | 0.000401068 |
| c.715C > A | p.Pro239Thr | rs1554769975 | missense_variant | 0.000398889 |
| c.755C > G | p.Pro252Arg | rs1303247012 | missense_variant | 0.00039776 |
| c.764T > C | p.Ile255Thr | rs1554770000 | missense_variant | 0.000397782 |
| c.773G > T | p.Arg258Eeu | rs138128200 | missense_variant | 0.000397807 |
| c.787C > G | p.Arg263Gly | rs782766545 | missense_variant | 0.000397842 |
| c.788G > C | p.Arg263Pro | rs535110977 | missense_variant | 0.000397905 |
| c.788G > T | p.Arg263Leu | rs535110977 | missense_variant | 0.003183091 |
| c.797T > C | p.Val266Ala | rs1554770013 | missense_variant | 0.003184105 |
| c.962A > G | p.Asp321Gly | rs1185288451 | missense_variant | 0.000408243 |

*Further studies showed that these variants do not find a unique match between the nucleotide sequence and the amino acid sequence among all the queried databases.
The above variants are, if not stated otherwise, extracted from the following database: https://gnomad.broadinstitute.org/gene/ENSG00000187134?dataset=gnomad_r2_1
¹identified in lipedema patients.
²These variants have been created in a mutagenesis experiment described by Couture et al.
³The enzyme activity parameters described by Couture et al. were used to calculate those of the first variant identified by Michelini et al. in a family with lipedema, p.(Leu213Gln).

The complete sequence of AKR1C1 and AKR1C2 genes are well known and documented in literature. The following links take to a database (https://www.ensembl.org/) that discloses details about both genes and whole sequences:

AKR1C1 gene summary:
https://www.ensembl.org/Homo_sapiens/Gene/Summary?db=core;g=ENSG00000187134;r=10:4963253-4983283
AKR1C1 transcript sequence (MANE select):
https://www.ensembl.org/Homo_sapiens/Transcript/Exons?db=core;g=ENSG00000187134;r=10:4963253-4983283;t=ENST00000380872
The sequence listing reports the complete sequence of the AKR1C1 gene (Homo sapiens) as SEQ ID NO 1, the corresponding coding sequence (cDNA) as SEQ ID NO 2 and two isoform corresponding proteins as SEQ ID NO 3 and SEQ ID NO 4. The DNA and corresponding protein sequence of the variant c.928A>C (p.(Ile310Leu)) are depicted as SEQ ID NO 5 and SEQ ID NO 6, respectively.
AKR1C2 gene summary:
https://www.ensembl.org/Homo_sapiens/Gene/Summary?db=core;g=ENSG00000151632;r=10:498777 5-5018031
AKR1C2 transcript sequence (MANE select):
https://www.ensembl.org/Homo_sapiens/Transcript/Exons?db=core;g=ENSG00000151632;r=10:4987 775-5018031;t=ENST00000380753
The sequence listing reports the sequence of the AKR1C2 gene (Homo sapiens) as SEQ ID NO 7, the corresponding exons as SEQ ID NO 8 and the protein corresponding to the exons as SEQ ID NO 9.

Further details regarding the identification of missense AKR1C1 variants in lipedema patients, sequencing, molecular modelling etc. are described in Michelini S, Chiurazzi P, Marino V, Dell'Orco D, Manara E, Baglivo M, Fiorentino A, Maltese P E, Pinelli M, Herbst K L, Dautaj A, Bertelli M., Aldo-Keto Reductase 1C1 (AKR1C1) as the First Mutated Gene in a Family with Nonsyndromic Primary Lipedema. Int J Mol Sci. 2020 Aug. 29; 21(17):6264. doi: 10.3390/ijms21176264. PMID: 32872468; PMCID: PMC7503355.

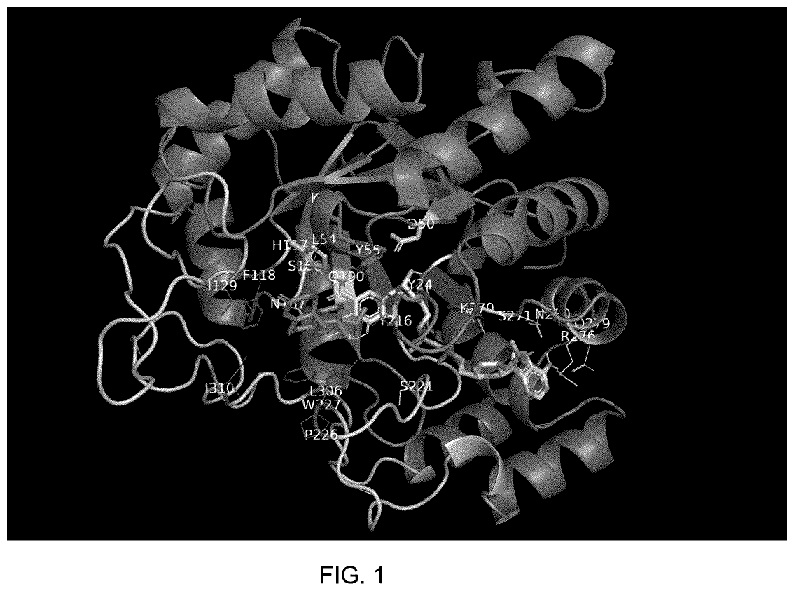

From structural analysis and molecular dynamics it was found that (FIG. 1): Human AKR1C1 three-dimensional structure shows an (αβ)8-barrel motif. Two more β-sheets B1 (7-9), B2 (15-17), and two more α-helices, H1(239-248) and H2 (290-298), not taking part in the core barrel structure. Three large loops complete the structure: loop A is located at 117-143, loop B is located at 217-238, and loop C is located at 299-322.

The NADP(H)-binding residues are highly conserved and include Thr23, Asp50, Ser166, Asn167, Gln190, Tyr216, Leu219, Ser221, Arg270, Ser271, Phe272, Arg276, Glu279 and Asn280, which contribute toward the binding affinity and specificity of the cofactor (see FIG. 1). Residues involved in substrate binding are: Tyr24, Leu54, Phe118, Phe129, Thr226, Trp227, Asn306 and Tyr310, while those involved in catalysis are: Asp50, Tyr55, Lys84 and His117 (see FIG. 1).

To describe the interaction of the enzyme with cofactor and substrate in energetic terms, thus to furnish an energy landscape of binding, molecular dynamics simulations were run on the AKR1C1/steroid/NADP(H) ternary complex, and binding energy was calculated as well by use of the MMPBSA (Genheden and Ryde, 2015) method and GROMACS molecular dynamics software (Abraham et al., 2015). The overall energies of binding for the two are (Table 3):

TABLE 3

| | | |
|---|---|---|
| Steroid (STR) | −115.4 kJ/mol | +/−10.6 kJ/mol |
| NADP(H) (NPD) | −337.6 kJ/mol | +/−53.9 kJ/mol |

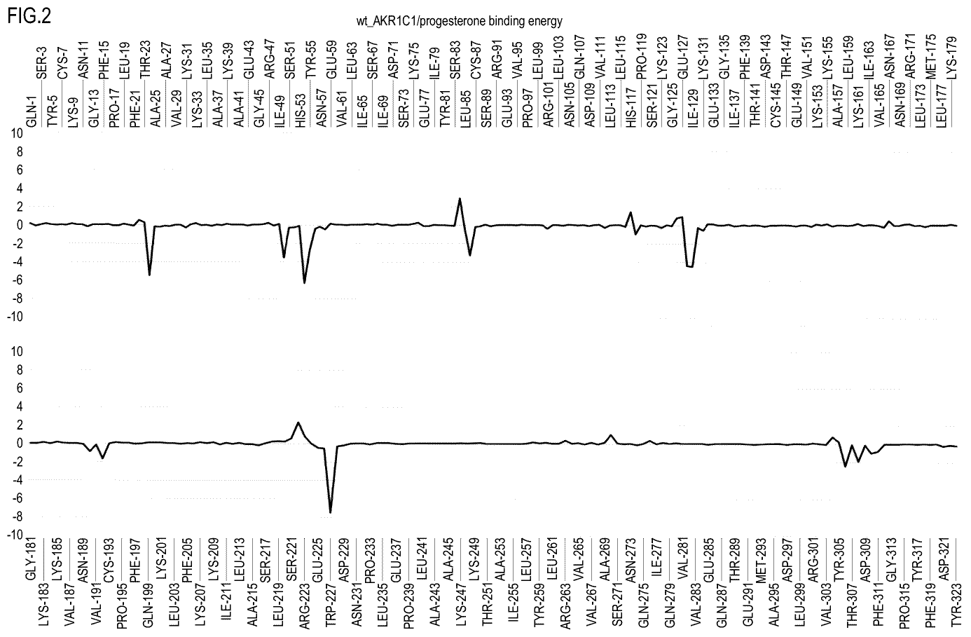

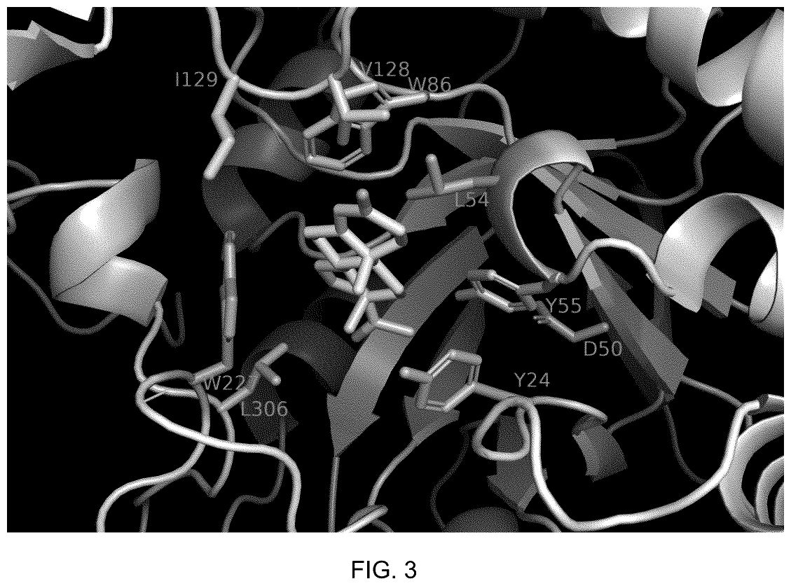

The MMPBSA method also allowed for quantification of the contribution to binding of each amino acid, allowing the impact of an amino acidic missense substitution to be evaluated as follows (see also Table 4). MMPSA profile of STR binding shows three amino acids account for 50% of the binding energy: Tyr24, Leu54, and Trp227; another significant contribution is given by Asp50, Tyr55, Trp86, Val128, Ile129, Leu306, showing an overall hydrophobic nature of the binding (see FIGS. 2 and 3).

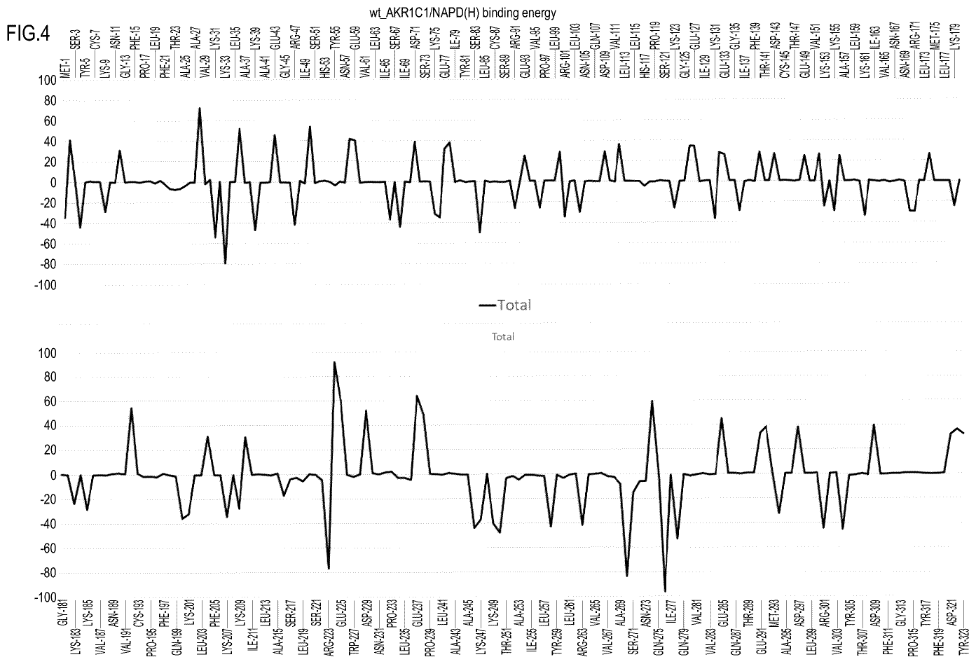

MMPBSA profile of NADP(H) binding is dominated by charge pairs giving prominent repulsion/attraction peaks between charged amino acids of the protein and the phosphate groups of the cofactor. The four most prominent negative binding energy peaks derive from Lys33, His222+Arg223, Lys270, Arg276, all neighboring the phosphate group on the 2′ position of the ribose ring that carries the adenine moiety (see FIG. 4). Such evidence accounts for the significant difference in affinity for NADP(H) vs NAD(H) cofactors in binding AKR enzymes, with the former showing a mid-nanomolar value (100 nM) whereas the latter binds with mid-micromolar affinity (200 mM).

Multiple alignments of protein sequences produce a matrix of aminoacids; by elaborating the columns as vectors, entropy of aminoacidic positions can be calculated according to Shannon, describing the amount of variability through a column in the alignment. The lower the value, the lower the variability accepted by the position. The inventors aligned 120 sequences from the AKR1C family to derive Shannon entropy (Strait & Dewey, 1996) of each position; values for each missense mutation from Table 2 (AKR1C1 selected variants) are reported in Table 4.

In silico mutagenesis of AKR1C1 and molecular dynamics simulations, entropy evaluation, binding energy for cofactor and for substrate allowed for the determination of the structural impact of variants, thus the structural consequence prediction on AKR1C1 that are conducive of loss function for many of the selected mutations in Table 2. Mutations are reported alongside their predicted effect in Table 4.

TABLE 4

| Transcript | | Shannon Entropy | | | |
|---|---|---|---|---|---|
| Consequence | | (natural | Interaction | Interaction | Predicted |
| AKR1C1: | Protein | value/normalized | with | with | structural |
| NM_001353.6: | Consequence | value to 4.32 max) | substrate | cofactor | consequence |
| c.160T > G | p.(Leu54Val)* | 2.11/0.49 | — | | Known to acquire the |
| | | | | | function of AKR1C2^{‡} |
| c.162A > T | p.(Leu54Phe)* | 2.11/0.49 | — | | Disruption of substrate binding |
| c.911G > T¹ | p.(Arg304Leu) | 0.61/0.14 | | | Disruption of folding |
| c.381A > T¹ | p.(Glu127Asp) | 1.72/0.40 | | | — |
| c.664_665delCAinsAT¹ | p.(His222Ile) | 1.78/0.41 | | — | Disruption of cofactor binding |
| c.664_665delCAinsTC¹ | p.(His222Ser) | 1.78/0.41 | | — | Disruption of cofactor binding |
| c.919_920delACinsGT¹ | p.(Thr307Val) | 3.21 /0.74 | | | — |
| c.925_926delGAinsCT | p.(Asp309Leu) | 2.95/0.68 | | | — |
| (p.Asp309Leu)¹ | | | | | |
| c.914A > T¹ | p.(Tyr305Phe) | 0.19/0.04 | | | — |
| c.638T > A² | p.(Leu213Gln)* | 0.26/0.06 | | | Disruption of folding |
| c.22G > C | p.(Val8Leu) | 1.07/0.25 | | | — |
| c.22G > T | p.(Val8Leu) | 1.07/0.25 | | | — |
| c.32A > G | p.(Asn111Ser) | 0.47/0.11 | | | — |
| c.97A > G | p.(Lys33Glu) | 1.95/0.45 | | — | Disruption of cofactor binding |
| c.104T > C | p.(Leu35Ser) | 2.98/0.69 | | | — |
| c.139C > A | p.(Arg47Ser) | 0.51/0.12 | | | Disruption of folding |
| c.163T > C | p.(Tyr55His) | 0/0 | — | | Disruption of catalysis |
| c.168T > A | p.(Asn56Lys) | 1.88/0.44 | | | — |
| c.184G > A | p.(Gly62Arg) | 0/0 | | | Disruption of folding |
| c.272G > T | p.(Arg91Leu) | 1.17/0.27 | | | Disruption of folding |
| c.274C > A | p.(Pro92Thr) | 0.33/0.08 | | | Disruption of folding |
| c.290C > G | p.(Pro97Arg) | 1.39/0.32 | | | Disruption of folding |
| c.298G > C | p.(Glu100Gln) | 0.14/0.03 | | | — |
| c.338T > C | p.(Leu113Pro) | 0/0 | | | Disruption of folding |
| c.355C > A | p.(Pro119Thr) | 0/0 | | | Disruption of folding |
| c.392A > T | p.(Lys131Ile) | 2.16/0.5 | | | — |
| c.394G > T | p.(Asp132Tyr) | 0.73/0.17 | | | — |
| c.566A > G | p.(Asn189Ser) | 0.14/0.03 | | | Disruption of folding |
| c.575A > C | p.(Glu192Ala) | 0/0 | | | Disruption of folding |
| c.616T > G | p.(Cys206Gly) | 0/0 | | | Disruption of folding |
| c.698C > T | p.(Pro233Leu) | 0.07/0.016 | | | Disruption of folding |
| c.715C > A | p.(Pro239Thr) | 0.12/0.03 | | | Disruption of folding |
| c.755C > G | p.(Pro252Arg) | 0.43/0.10 | | | Disruption of folding |
| c.764T > C | p.(Ile255Thr) | 0.97/0.22 | | | Disruption of folding |
| c.773G > T | p.(Arg258Leu) | 0.07/0.016 | | | Disruption of folding |
| c.787C > G | p.(Arg263Gly) | 0.24/0.06 | | | Disruption of folding |
| c.788G > C | p.(Arg263Pro) | 0.24/0.06 | | | Disruption of folding |
| c.788G > T | p.(Arg263Leu) | 0.24/0.06 | | | Disruption of folding |
| c.797T > C | p.(Val266Ala) | 0.12/0.03 | | | Disruption of folding |
| c.962A > G | p.(Asp321Gly) | 1/0.23 | | | — |
| c.840C > A | p.(Asn280Lys)* | 0.21/0.05 | | — | Disruption of cofactor binding |
| c.327T > A | p.(Asp109Glu)* | 0.38/0.09 | | | — |
| c.928A > C³ | p.(Ile310Leu)* | 2.93/0.68 | | | — |

Legend.
*Variants found in lipedema families are marked with an asterisk and a detailed description of structural consequences is reported below;
^{‡}references: (Penning et al., 2019; Hara et al., 1996; Matsuura et al., 1997).
The above variants are, if not stated otherwise, extracted from the following database: https://gnomad.broadinstitute.org/gene/ENSG00000187134?dataset=gnomad_r2_1
¹These variants have been created in a mutagenesis experiment described by Couture et al.
²The enzyme activity parameters described by Couture et al. were used to calculate those of the first variant identified by Michelini et al. in a family with lipedema, p.(Leu213Gln).
³This variant is not described in the above database, the respective DNA and protein sequences are reflected by SEQ ID NO 5 and 6, respectively.

In the following the Applicant reports a detailed descriptions of structural consequences of variants found in lipedema families.

In the inventors' patients, four missense mutations were found, namely Leu54Val, Leu54Phe, Asn280Lys, and Leu213Gln. The effects of such mutations on enzyme folding, stability, and biological activity have been studied with structural biology, and molecular dynamics approach to evaluate their involvement in lipedema development.

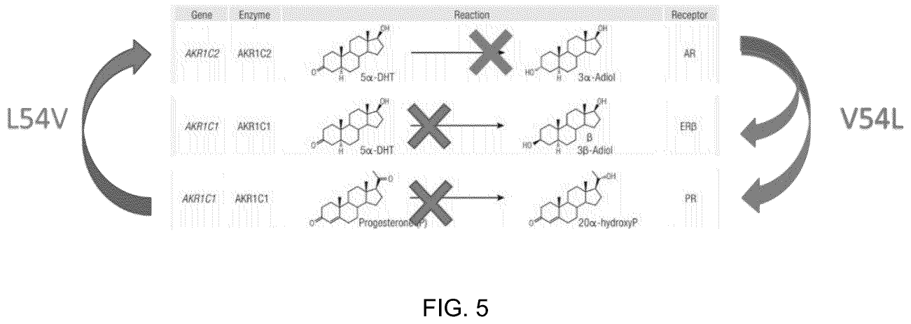

Starting with Leu54Val and Leu54Phe, the role of Leu54 in substrate selectivity has been already elucidated (Penning et al., 2019; Hara et al., 1996; Matsuura et al., 1997), and can be summarized as follows (see also FIG. 5). Human AKR1C1 and AKR1C2 differ in that AKR1C1 exhibits 20α-HSD activity, whereas AKR1C2 exhibits 3α-HSD. The two enzymes differ for seven amino acids, and only one is located at the active site at position 54: leucine for C1 and valine for C2. The replacement of Leu54 by the less bulky valine changes the 20α activity to 3α. Consistently, the reverse mutation Val54Leu converts the 3α-HSD into 20α-HSD regarding its activity (Zhang et al., 2014). Evidence that enzymes work in the reduction direction in mammalian cells (Byrns et al., 2010; Byrns et al., 2012; Rizner et al., 2003; Rizner et al., 2006) lead the Leu54Val mutation to hamper the processing of progesterone.

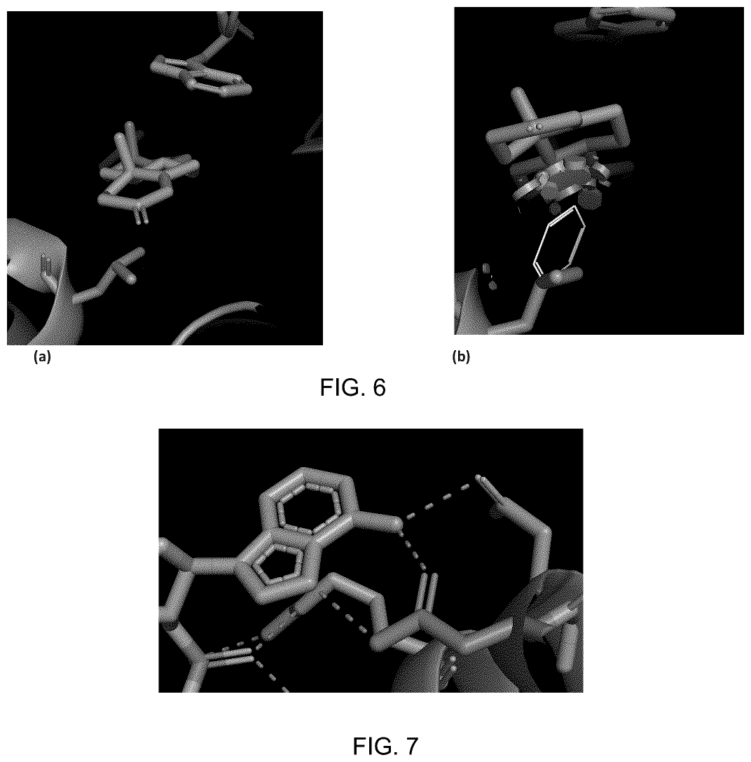

Similarly, the interaction between the steroid and the enzyme is disrupted by the replacement of the same Leu54 by phenylalanine, as shown by the molecular dynamics simulation. In the wildtype, Leu54 and Trp227 play a significant role in binding the steroid by interacting with opposite faces of the polycyclic ring of the ligand and contribute as much as 33% of the overall binding energy. Mutation of Leu54 to Phe, although enhancing the hydrophobic nature of the interaction, introduce a second large, aromatic sidechain in place hampering the ligand entrance in the site and conducive of binding disruption (see FIG. 6 (a) and (b)). Indeed, from the molecular dynamics simulations, we noticed that the steroid was unstable, and phenylalanine was pushed back.

Interestingly, phenylalanine is present at position 54 in the wildtype, non-human AKR1C8P, but here the steric hindrance with the opposite amino acid 227 is compensated by the presence of the smaller asparagine. At the same time, the cumbersome tryptophan is ‘shifted’ to position 228. Nonetheless, 1C8 preserve the same 20α-HSD activity of 1C1. As previously mentioned, this may indicate coevolution between positions 54 and 227.

Referring now to Asn280Lys, it can be said that asparagine 280 takes part in cofactor binding; together with Gln279 it is responsible for adenine group binding through a hydrogen bond to the amine group (see FIG. 7). The molecular simulation showed how Asn is the stronger binder of the two. Such finding is also confirmed by the molecular mechanics' energy contributions to the cofactor binding resulting from MMPBSA, showing 6-fold higher interaction energy for Asn280 with respect to Gln279 (17 kJ/mol vs. 3 kJ/mol).

Although such variant involved the replacement of a small side chain with a bulky one, molecular modelling showed how hydrophobic moiety of lysine can be easily accommodated by displacement of water molecules. MD simulation confirmed a small effect is exerted on the protein structure, while the missing H-bond acceptor capability of Lys led to the loss of interaction with the adenine ring, resulting in the aromatic ring flipping away from its position, also because of the attraction of Lys280 to the phosphate group. The optimal binding geometry is then disrupted rather than folding.

AKR1C1 is a member of the AKR1C family of enzymes that share a high percentage of amino acid sequence identity (from 84 to 98%). This family catalyzes NADPH dependent oxydoreductions either for the biosynthesis or inactivation of steroid hormones, bile acids and neurosteroids. All AKR1C enzyme catalyze a sequential ordered Bi-Bi substrate enzyme reaction. In particular, AKR1C1 in involved in the “alternative pathway” of androgen biosynthesis inactivating the most potent androgen 5alpha-dihydrotestosterone (5alpha-DHT) to 5alpha-androstane-3beta,17beta-diol, a potent agonist of ERbeta which exerts anti-proliferative effect. Androgens play an important role in regulation of body fat distribution in humans. They exert direct effects on adipocyte differentiation in a depot-specific manner, via the androgen receptor (AR), leading to modulation of adipocyte size and fat compartment expansion. AKR1C1 can also regulate the cellular concentration of allopregnanolone by preventing its formation from progesterone and by catalyzing its inactivation. Indeed, AKR1C1 catalyzes progesterone reaction to form the less potent progestogen 20alpha-hydroxy-4-pregnen-3-one, reduce 5alpha-pregnane-3,20-dione (5alpha-DHP) to form 20alpha-hydroxy-5alpha-pregnan-3-one or 3alpha-hydroxy-5alpha-pregnan-20-one (allopregnanolone) to a less neuroactive 5alpha-pregnane-3alpha,20alpha-diol. AKR1C1 therefore is involved in the inactivation of allopregnanolone, that acts in the central nervous system as positive allosteric modulator of gamma aminobutyric acid receptor A (GABAA). As other enzyme of the family can reduce also 20alpha-hydroxy-5alpha-pregnan-3-one to 5alpha-pregnane-3alpha,20alpha-diol. Progesterone has lipogenic action on adipose tissue by upregulating adipocyte determination and differentiation through 1/sterol regulatory element-binding protein 1c (ADD1/SREBP1c) expression in primary cultured preadipocyte from rat parametrial adipose tissue (Lacasa et al., 2001). ADD1/SREBP1c promotes adipocyte differentiation and gene expression linked to fatty acid metabolism (Kim and Spiegelman, 1996). The levels of progesterone and 5alpha-dihydrotestosterone can be detected in body fluids. Levels of progesterone ranges during normal menstrual cycles from 0 ng/ml (follicular phase) to 28 ng/ml (central luteal phase), values range from 11 to 422 ng/ml during pregnancy, while in post menopause or in males, levels of progesterone are less than 1.2 ng/ml. Levels of 5alpha-DHT range from 250-990 pg/ml in males, from 24-368 in pre-menopause females and from 10-181 in post menopause females.

A recent study revealed that the best combination to diagnose polycystic ovary syndrome (PCOS), including up to four steroids, was a ratio comprising androstanediol, estriol, 20βDHcortisone and cortisol accordingly to the following formula: (androstanediol^{1.5}×20β-DH-cortisone)/(20β-DH-cortisone+[cortisol×log(estriol)]. This ratio was significantly increased in PCOS compared to controls at a threshold value of ≥435 (Dhayat et al., 2018). Considering the activity of the AKR1C1 enzyme, this ratio reasonably has diagnostic value in lipedema.

AKR1C1 is also involved in catalyzing the synthesis of prostaglandins in humans (Dozier et al., 2008). It has been shown that prostaglandin 2 alpha (PGF2α) inhibited adipogenesis by activating at its specific receptor on preadipocytes (Lepak and Serrero, 1995; Taketani et al., 2014). In mice, a decrease in intra-adipose tissue PGF2α levels following Akr1b7 ablation leads to increased adiposity, a phenotype that is reversed by the chronic administration of Cloprostenol, a PGF2α agonist (Volat el al., 2012). PGF2α and its derivatives can therefore be used as molecular diagnostic/prognostic markers and therapeutic agents also in lipedema. PGF2α can be reliably quantified by measurement of 15-keto-13,14-dihydro-PGF2α, the major metabolite of PGF2α in plasma (Helmersson et al., 2005). The isoprostane 8-iso-Prostaglandin F2α (8-iso-PGF2α), a prostaglandin-like molecule, is a quantitative ROS biomarker used to measure oxidative stress in vivo which correlates positively with BMI, intra-abdominal fat and waist circumference (Milne et al., 2015; Jia et al., 2019). Both molecules can be easily quantified in different body fluids such as plasma, serum or urine.

A list of AKR1C1 metabolites for use in diagnostics is reported in table 5.

TABLE 5
AKR1C1 metabolites for use in diagnostics

| Molecule | Common name |
|---|---|
| 3α-Hvdroxy-5α-pregnan-20-one | Allopregnanolone (allo) |
| 3α-Hydroxy-5β-pregnan-20-one | Pregnanolone (preg) |
| 3β-Hvdroxy-5α-pregnan-20-one | Isopregnanolone (iso) |
| 3β-Hydroxy-5β-pregnan-20-one | Epipregnanolone (epi) |
| 5α-Pregnane-3,20-dione | 5α-Dihydroprogesterone (5α-DHP) |
| 5β-Pregnane-3,20-dione | 5β-Dihydroprogesterone (5β-DHP) |
| Pregn-4-ene-3,20-dione | Progesterone (P) |
| 20α-Hydroxy-pregn-4-ene-3-one | 20α-dihydroprogesterone (20α-OHP) |
| 5α-Pregnane-3α,20α-diol | Allopregnanediol |
| 5β-Pregnane-3α,20α-diol | Pregnanediol |
| 5α-Androstan-17β-ol-3-one | 5α-Dihydrotestosterone (5α-DHT) |
| 5α-androstane-3α,17β-diol | 3α-Androstanediol (3α-Adiol) |
| 21-hydroxy-5α-pregnan-20-one | 5α-Dihydrodeoxycorticosterone (5αDHDOC) |
| 3a,21-dihydroxy-5α-pregnan-20-one | 3α,5α-Tetrahydrodeoxycorticosterone |
| Pregnanetriol/17-hydroxypregnanolone | (alloTHDOC) (P3)/(17HP) |
| 15-keto-13,14-dihydro-PGF2α | PGFM |
| 8-iso-Prostaglandin F2α | 8-iso-PGF2α |

In the literature, a number of natural and synthetic compounds are known to exert a modulatory action on the key human progesterone-metabolizing enzyme, AKR1C1.

A list of compounds for treatments for lipedema comprising the use of natural molecules or chemicals that modulate the catalytic activity of the AKR1C1 enzyme are shown in table 6.

TABLE 6
Natural and synthetic compounds that modulate AKR1C1

| | | Enzyme activity |
|---|---|---|
| Compound | Main sources | (inhibition/activation) |
| 2,3-dimethoxynaphthalene-1,4-dione (DMNQ) | Synthetic | activation |
| 20α-hydroxydydrogesterone | Synthetic | inhibition |
| 3,5-dichlorosalycilic acid | Synthetic | inhibition |
| 3,5-diiodosalycilic acid | Synthetic | inhibition |
| 3,7-dihydroxyflavone | Synthetic | inhibition |
| 3-bromo-5-phenylsalicylic acid | Synthetic | inhibition |
| 3-Hihydroxy flavone | Synthetic | inhibition |
| 5-Hihydroxy flavone | Synthetic | inhibition |
| 5,7-Dihydroxyflavone | Passiflora coerulea | inhibition |
| 5-Metoxy flavone | Synthetic | inhibition |
| 7-Hydroxy flavone | Synthetic | inhibition |
| Abietic acid | Pine wood | inhibition |
| AKR1C1 Inhibitor, 5-PBSA | Synthetic | inhibition |
| AKR1C1-IN-1 | Synthetic | inhibition |
| Apigenin | Snapdragon, chamomille | inhibition |
| Benzodiazepines (diazepam, | Synthetic | inhibition |
| medazepam, estazolam, flunitrazepam, | | |
| nitrazepam, cloxazolam, bromazepam, | | |
| oxazolam and oxazepam) | | |
| Biochanin A | Red clover, soy, alfalfa sprouts, | inhibition |
| | peanuts, chickpea (Cicer | |
| | arietinum) and in other legumes | |
| Chrysin | Scutellaria baicalensis | inhibition |
| Coumarin | Woodruff, vanilla, lavender oil, | inhibition |
| | tonka bean, minor constituent in | |
| | cherries, strawberries, apricots | |
| Coumestrol | Soybeans, brussels sprouts, | inhibition |
| | spinach and a variety of legumes, | |
| | clover, Kala Chana, Alfalfa sprouts | |
| Cyclopentanone | Synthetic | inhibition |
| Curcumin | Curcuma longa | Unknown |
| Daidzein | Soybeans, beer | inhibition |
| Diethylstilbestrol | Synthetic | inhibition |
| Dydrogesterone | Synthetic | inhibition |
| Equilin | Horse estrogen; estrogen | inhibition |
| | replacement therapy | |
| Ethacrynic acid | Synthetic | activation |
| Flavanone | yellow/red fruits, vegetables | inhibition |
| Flavone | yellow/red fruits, vegetables | inhibition |
| Genistein | Soybeans, beer | inhibition |
| Glycyrrhetinic acid | Licorice | inhibition |
| Hydrogen peroxide | Synthetic | activation |
| Kaempferol | Tea, grapes, berries and | inhibition |
| | cruciferous vegetables | |
| Liquiritin | Licorice | inhibition |
| Luteolin | Parsley, artichoke, basil, celery | inhibition |
| Mangosteen extract | Mangosteen | inhibition |
| Medroxyprogesterone acetate | Synthetic | inhibition |
| Methyl jasmonate | Derived from jasmonic acid as | inhibition |
| | found in many plants | |
| Naringenin | Grapefruit | inhibition |
| Nonsteroidal Anti-Inflammatory Drugs | Synthetic | inhibition |
| (mefenamic acid, indomethacin, | | |
| celecoxib, diclofenac, naproxen, | | |
| ibuprofen, ketoprofen, paracetamol, | | |
| acetylsalicylic acid, etodolac, 3- | | |
| phenoyxbenzoic acid, sulindac, | | |
| meclofenamic acid, zomepirac, | | |
| Norethinodrone | Synthetic | inhibition |
| Quercetin | Chamomille, red onions, apples, | inhibition |
| | tea, endive | |
| Resveratrol | Skins of certain red, grapes, in | inhibition |
| | peanuts, blueberries, pines, roots and | |
| | stalks of knotweed | |
| Steroidal Inhibitors | Synthetic | inhibition |
| (medroxyprogesterone acetate, | | |
| bethamethasone, steroidal lactones, | | |
| cholanic acid derivatives | | |
| t-butylhydroquinone | Synthetic | activation |
| Tamoxifen | Synthetic | inhibition |
| Wagonin | Scutellaria baicalensis | inhibition |
| Zearalenone | Mold-infected grain and feeds | inhibition |

PGE2 and PGF2α and its analogue (viprostol, latanoprost, isopropyl unoprostone, bimatoprost) can exhibit antiadipogenic properties. Some active constituents from Chinese herbs as ricinoleic acid, acteoside, amentoflavone, quercetin-3-O-rutinoside and hinokiflavone were predicted to be prostaglandin D2 synthase (PTGDS) inhibitors (Fong et al., 2015). Inversely, other natural supplements such as chlorella and green tea are proposed be used to decrease PGE2 and PGF2α levels (Koeberle et al., 2009; Haidari et al., 2018).

A list of compounds for treatments of lipedema comprising the use of natural molecules or chemicals that modulate prostaglandins are shown in table 7.

TABLE 7
Natural and synthetic compounds that modulate prostaglandins

| Compound | Main sources | Activity |
|---|---|---|
| Acteoside | Rehmannia glutinosa | PTGDS inhibitors |
| Amentoflavone | Biota orientalis | PTGDS inhibitors |
| Chlorella | Chlorella | decrease PGE2 and PGF2α levels |
| Green tea | Green tea | decrease PGE2 levels |
| Hinokiflavone | Platycladus orientalis | PTGDS inhibitors |
| Quercetin-3-O-rutinoside | Platycladus orientalis | PTGDS inhibitors |
| Ricinoleic acid | Ricinus communis | PTGDS inhibitors |
| Sennosides | Cassia species | increase PGE2 formation |
| Viprostol, latanoprost, isopropyl | Syntetic | PGF2α analogues |
| unoprostone, bimatoprost | | |

Natural and synthetic compounds that modulate AKR1C1 and listed in Table 6 were submitted to molecular docking procedure by using Autodock Vina 1.2 with the following parameters: AKR1C1 and NADPH coordinate from PDB entry 1MRQ; amino acids Tyr24, Leu54 and Trp227 set as flexible sidechain: docking box set centered at x=4.29 y=33.9 z=17.06 with size x=17.39 y=11.16 z=12.67, vina scoring function. Results are reported as binding affinity in Kcal/mol (the lowest, the better) in Table 8. Taking 2 Kcal/mol as the common threshold for binding energy significance, we have 11 top compounds (in bold); significantly 6 out of 11 are simple flavones/flavonones (in bold and italics). The double stacking interaction of B ring with Tyr24 phenol and Trp227 indole rings is the driving force of the interaction.

TABLE 8
Docking analysis of natural and synthetic
compounds that modulate AKR1C1

| | Binding affinity |
|---|---|
| Compound | (Kcal/mol) |
| Flavanone | −17.45 |
| Flavone | −17.25 |
| 3-Hydroxyflavone | −16.12 |
| 5-Hydroxyflavone | −16.09 |
| Equilin | −15.84 |
| Diazepam | −15.70 |
| 20α-Hydroxydydrogesterone | −15.69 |
| Coumarin | −15.58 |
| Glycyrrhetinic Acid | −15.57 |
| 7-Hydroxyflavone | −15.54 |
| 3,7-Dihydroxyflavone | −15.48 |
| Coumestrol | −15.20 |
| Apigenin | −15.16 |
| Flurbiprofen | −15.06 |
| Abietic Acid | −15.04 |
| Mefenamic Acid | −15.03 |
| Beta-Mangostin | −15.00 |
| Cholanic Acid | −14.98 |
| Alpha-Mangostin | −14.96 |
| 5,7-Dyhydroxyflavone | −14.94 |
| Naringenin | −14.93 |
| Ketoprofen | −14.91 |
| Naproxen | −14.70 |
| Luteolin | −14.70 |
| Quercetin | −14.65 |
| Gamma-Mangostin | −14.62 |
| Norethindrone | −14.60 |
| Betamethasone | −14.51 |
| Biochanin A | −14.43 |
| Oxazolam | −14.42 |
| Medazepam | −14.42 |
| 3-Bromo,5-Phenylsalicylic Acid | −14.32 |
| 5-Pbsa | −14.32 |
| Genistein | −14.28 |
| Liquiritin | −14.18 |
| Meclofenamic Acid | −14.17 |
| Sulindac | −14.09 |
| 5-Methoxyflavone | −14.06 |
| Zearalenone | −13.98 |
| Nitrazepam | −13.96 |
| Estazolam | −13.85 |
| Kaempferol | −13.85 |
| Spironolactone | −13.75 |
| Wagonin | −13.74 |
| Bromazepam | −13.73 |
| Indomethacin | −13.71 |
| Daidzein | −13.68 |
| Oxazepam | −13.66 |
| Paracetamol | −13.66 |
| Resveratrol | −13.50 |
| Medroxyprogesterone Acetate | −13.46 |
| 2,3-Dimethoxynaphthalene-1,4-Dione | −13.43 |
| Cloxazolam | −13.40 |
| Medroxyprogesterone Acetate | −13.33 |
| Diethylstilbestrol | −13.31 |
| Ibuprofen | −13.23 |
| Cyclopentanone | −13.20 |
| Zomepirac | −13.03 |
| 3,5-Dichlorosalicylic Acid | −12.97 |
| Flunitrazepam | −12.93 |
| Tamoxifen | −12.89 |
| Hydroxytyrosol | −12.75 |
| T-Butylhydroquinone | −12.60 |
| Curcumin | −12.52 |
| Ethacrynic Acid | −12.37 |
| Methyl Jasmonate | −11.84 |
| 3,5-Diiodosalicylic Acid | −11.76 |

The analysis has been repeated for AKR1C1 mutant Leu54Val by using the same parameters (Table 9). Such mutation is known to convert enzymatic activity of AKR1C1 into that of AKR1C2, which might eliminate androgen inhibitory effects on adipogenesis favouring progression of adipogenesis (Kiani et al., 2021), thus selective targeting of such mutation would modulate its possible effect on fat deposition. Again, flavones are among the favorites, but with lower affinity and competing with natural steroids like equilin or with large pentacyclic molecules glycyrrhetinic acid; this is due to the lower steric hindrance of valine vs. leucine resulting in less selective active site.

TABLE 9
Docking analysis of natural and synthetic compounds
interacting with AKR1C1 mutant Leu54Val

| | Binding affinity |
|---|---|
| Compound | (Kcal/mol) |
| Glycyrrhetinic Acid | −14.41 |
| Equilin | −14.22 |
| Flavone | −14.18 |
| Flavanone | −14.11 |
| 3-Hydroxyflavone | −14.10 |
| Abietic Acid | −13.91 |
| Coumestrol | −13.63 |
| Nitrazepam | −13.59 |
| Betamethasone | −13.52 |
| 5-Hydroxyflavone | −13.49 |
| 20α-Hydroxydydrogesterone | −13.46 |
| Diazepam | −13.42 |
| Cholanic Acid | −13.41 |
| Alpha-Mangostin | −13.41 |
| Gamma-Mangostin | −13.38 |
| Beta-Mangostin | −13.34 |
| Estazolam | −13.29 |
| Genistein | −13.24 |
| Sulindac | −13.08 |
| 5-Pbsa | −13.04 |
| 3-Bromo-5-Phenylsalicylic Acid | −13.04 |
| 3,7-Dihydroxyflavone | −13.00 |
| Oxazepam | −12.93 |
| Norethindrone | −12.84 |
| 7-Hydroxyflavone | −12.84 |
| Naringenin | −12.84 |
| Daidzein | −12.80 |
| 5-Methoxyflavone | −12.72 |
| 5,7-Dyhydroxyflavone | −12.62 |
| Flunitrazepam | −12.61 |
| Bromazepam | −12.59 |
| Celecoxib | −12.59 |
| Zomepirac | −12.58 |
| Apigenin | −12.57 |
| Luteolin | −12.56 |
| Kaempferol | −12.51 |
| Quercetin | −12.47 |
| Medroxyprogesterone-Acetate | −12.41 |
| Medroxyprogesterone-Acetate | −12.41 |
| Coumarin | −12.40 |
| Wagonin | −12.39 |
| Zearalenone | −12.35 |
| Indomethacin | −12.30 |
| Meclofenamic Acid | −12.24 |
| Biochanin-A | −12.17 |
| Flurbiprofen | −12.11 |
| Ketoprofen | −12.07 |
| Liquiritin | −12.06 |
| Diclofenac | −12.05 |
| Naproxen | −12.04 |
| Mefenamic Acid | −11.94 |
| Medazepam | −11.69 |
| 3-Phenoxybenzoic Acid | −11.55 |
| Resveratrol | −11.50 |
| Oxazolam | −11.46 |
| Cloxazolam | −11.42 |
| Diethylstilbestrol | −11.39 |
| Spironolactone | −11.33 |
| Etodolac | −11.33 |
| Tamoxifen | −11.18 |
| Hydroxytyrosol | −11.08 |
| Methyl-Jasmonate | −10.83 |
| T-Butylhydroquinone | −10.79 |
| Cyclopentanone | −10.69 |
| Curcumin | −10.61 |
| Ethacrynic Acid | −10.57 |
| 2,3-Dimethoxynaphthalene-1,4-Dione | −10.42 |
| Ibuprofen | −10.24 |
| Paracetamol | −10.15 |
| Acetylsalicylic Acid | −10.12 |
| 3,5-Diiodosalicylic Acid | −10.02 |
| 3,5-Dichlorosalicylic Acid | −9.69 |

AKR1C1 Leu54Phe mutant is the other variant affecting substrate binding site accessibility presently analyzed. Oppositely but coherently with Leu54Val flavones are the tighter binders due to the incremented steric hindrance of phenylalanine which is able to stacking interact with A/C rings of the binder (Table 10).

TABLE 10
Docking analysis of natural and synthetic compounds
interacting with AKR1C1 mutant Leu54Val

| | Binding affinity |
|---|---|
| Compound | (Kcal/mol) |
| Flavone | −18.35 |
| Flavanone | −18.21 |
| Medroxyprogesterone-Acetate | −18.04 |
| Cholanic Acid | −17.96 |
| Equilin | −17.81 |
| Estazolam | −17.48 |
| 5-Hydroxyflavone | −17.29 |
| Nitrazepam | −17.29 |
| Diazepam | −17.11 |
| 7-Hydroxyflavone | −16.94 |
| 20α-Hydroxydydrogesterone | −16.81 |
| Zearalenone | −16.76 |
| Spironolactone | −16.45 |
| 3-Hydroxyflavone | −16.42 |
| Medazepam | −16.35 |
| Cloxazolam | −16.33 |
| Norethindrone | −16.30 |
| Sulindac | −16.24 |
| Glycyrrhetinic Acid | −16.17 |
| Ketoprofen | −16.15 |
| 5,7-Dyhydroxyflavone | −16.13 |
| 5-Pbsa | −16.05 |
| 3-Bromo-5-Phenylsalicylic Acid | −16.05 |
| Betamethasone | −15.93 |
| Daidzein | −15.91 |
| Gamma-Mangostin | −15.77 |
| Naproxen | −15.72 |
| 3-7-Dihydroxyflavone | −15.72 |
| Coumestrol | −15.70 |
| Apigenin | −15.68 |
| T-Butylhydroquinone | −15.66 |
| Luteolin | −15.60 |
| Naringenin | −15.56 |
| Genistein | −15.54 |
| Celecoxib | −15.51 |
| Bromazepam | −15.51 |
| Resveratrol | −15.46 |
| Oxazolam | −15.46 |
| Liquiritin | −15.46 |
| Abietic Acid | −15.44 |
| Coumarin | −15.43 |
| Alpha-Mangostin | −15.27 |
| 3-Phenoxybenzoic Acid | −15.24 |
| Etodolac | −15.23 |
| 3,5-Dichlorosalicylic Acid | −15.23 |
| Biochanin-A | −15.22 |
| Flurbiprofen | −15.21 |
| Diethylstilbestrol | −15.12 |
| Wagonin | −15.09 |
| Oxazepam | −15.02 |
| Flunitrazepam | −15.01 |
| Kaempferol | −14.96 |
| 5-Methoxyflavone | −14.87 |
| Mefenamic Acid | −14.82 |
| Zomepirac | −14.57 |
| Beta-Mangostin | −14.56 |
| 3,5-Diiodosalicylic Acid | −14.46 |
| Acetylsalicylic Acid | −14.34 |
| Methyl-Jasmonate | −14.09 |
| Paracetamol | −13.97 |
| Diclofenac | −13.92 |
| Quercetin | −13.88 |
| Hydroxytyrosol | −13.86 |
| Indomethacin | −13.80 |
| Meclofenamic Acid | −13.78 |
| 2,3-Dimethoxynaphthalene-1,4-Dione | −13.73 |
| Ibuprofen | −13.58 |
| Cyclopentanone | −13.29 |
| Curcumin | −13.00 |
| Tamoxifen | −12.89 |
| Ethacrynic Acid | −12.19 |

The molecules of tables 9 and 10 have been analyzed considering the interaction with two specific variants, both on nucleotide 54, of particular interest is the Leu54Val variant. For every substance indicated in table 8, it is possible to identify through a study determining the affinity to AKR1C1 the most efficient one for a patient with a specific variant, as done for a patient with a variant on nucleotide 54.

Turning now to the surprising discovery of the inventors that AKR1C2 overexpression is a frequent feature of lipedema, a cohort of 19 lipedema patients and 2 affected family members from the family previously described in [Michelini et al., 2020] were enrolled in the study (Table 11).

TABLE 11
Clinical data of 19 lipedema patients

| Clincial data | | 19 F | |
|---|---|---|---|
| Mean age | | 42.8 | ±10.5 |
| Familiarity | | 100% | |
| Onset | childhood | 1 | 5.3% |
| | puberty | 14 | 73.7% |
| | adulthood (>20 yrs) | 3 | 15.8% |
| | Not Known | 1 | 5.2% |
| Localization | buttocks | 16 | 84.2% |
| of fat depots | legs | 15 | 78.9% |
| | thighs | 19 | 100% |
| | arms | 12 | 63.2% |
| | forearms | 4 | 21.5% |
| | trunk and abdomen | 1 | 5.2% |

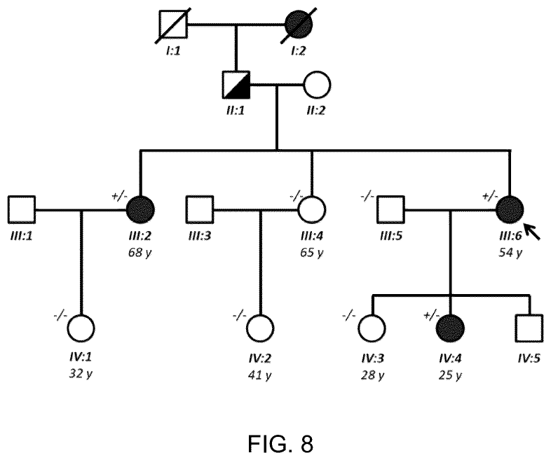

The L213Q family tree of the inventors' previous study [Michelini et al., 2020] is represented in FIG. 8. An AKR1C2 mRNA overexpression was detected in three affected family members (the patient bearing the L213Q variant and two of her relatives).

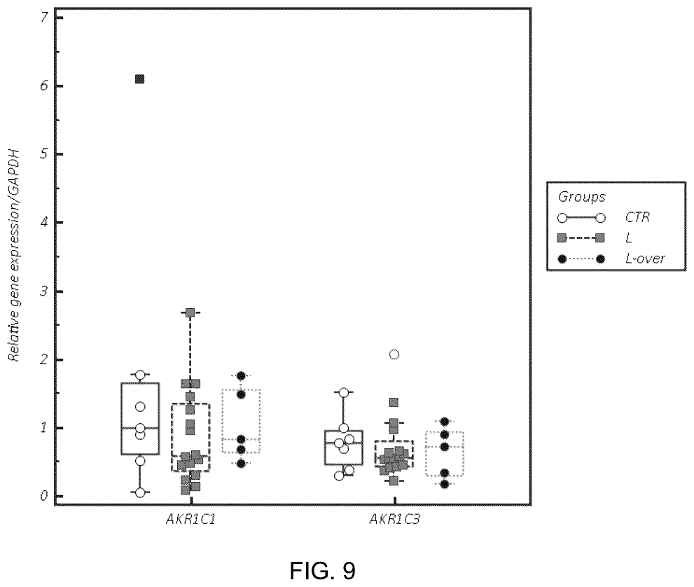

qPCR analysis was performed on blood RNA extracted from a pool of 21 patients with lipedema (Table 12, FIG. 8) and 7 healthy female controls. While AKR1C1 and AKR1C3 expression in blood was not different between groups, AKR1C2 expression was high in few lipedema patients (N=5), including 3 affected family members from our previously described AKR1C1 L213Q mutated family. Relative expression is depicted in FIG. 9, that shows the relative expression of AKR1C1 and AKR1C3 in different groups (CTR=non affected controls, L=lipedema patients without overexpression of AKR1C2, L-over=Lipedema patients with overexpression of AKR1C2), showing that lipedema patients expressed AKR1C1 and AKR1C3 levels similar to the control group.

For the qPCR analysis total RNA was extracted from blood using the Tempus™ Spin RNA Isolation Kit following manufacturer protocols. The SuperScript VILO cDNA Synthesis Kit was used to generate first strand cDNA. Quantitative real-time polymerase chain reaction (qPCR) was performed by using the PowerUp™ SYBR™ Green Master Mix (Thermofisher) on a QuantStudio 3 Real-Time PCR Systems. The primers used in the qPCR experiments were previously described and are the following:

(SEQ ID NO 10)
GACAAGCTTCCCGTTCTCAG;
and
(SEQ ID NO 11)
GGAGTCAACGGATTTGGTCG
for GAPDH;
(SEQ ID NO 12)
CCTAAAAGTAAAGCTTTAGAGGCCACC,
and
(SEQ ID NO 13)
GAAAATGAATAAGGTAGAGGTCAACATAAT
for AKR1C1,
(SEQ ID NO 14)
CCTAAAAGTAAAGCTCTAGAGGCCGT,
and
(SEQ ID NO 15)
GAAAATGAATAAGATAGAGGTCAACATAG
for AKR1C2,
(SEQ ID NO 16)
GAGAAGTAAAGCTTTGGAGGTCACA,
and
(SEQ ID NO 17)
CAACCTGCTCCTCATTATTGTATAAATGA
for AKR1C3 [Zhang et al., 2014;
Penning, Trevor M., 1997].

In two additional patients, two other variants were found (Table 12).

TABLE 12
Variants found in additional lipedema patients,
rs143258520 concerns a regulatory region upstream AKR1C1,
while NP_995317.1:p.Ser320PhefsTer2 concerns
the C-term removal of AKR1C2, predicted to increase
the binding affinity for DHT.

| Variant ID | Type |
|---|---|
| rs143258520 | AKRC1 downstream |
| NP_995317.1:p.Ser320PhefsTer2 | AKR1C2 c-term removal |

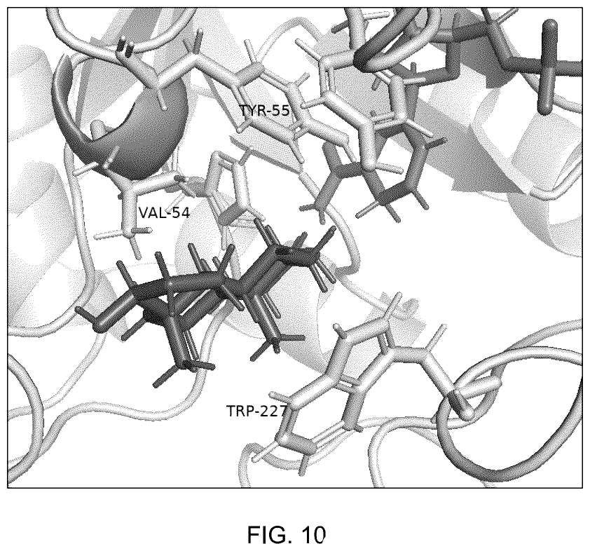

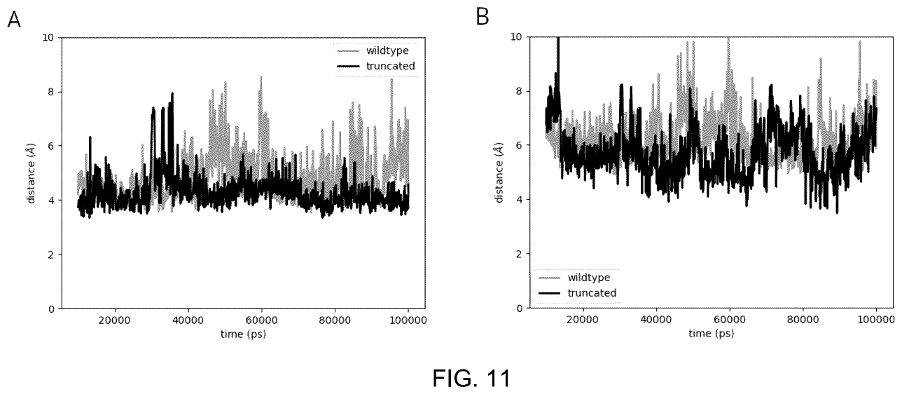

One included a variant in the regulatory region downstream AKR1C1, which may affect the relative expression levels between AKR1 C1 and AKR1C2; on the other hand, the other variant consisted in the c-term removal of AKR1C2, which is reported to affect DHT reduction rate in pig AKR1C1. The C-terminal region significantly contributes to the NADPH-dependent reductase activity for the 5α-DHT reduction [Son et al., 2015], an activity reserved for AKR1C2 in humans. We performed molecular dynamics study to evaluate the effect of this deletion. The results show that DHT adapts a more stable conformation in the truncated protein, where it is properly sandwiched between Val54 and Trp227, with Trp227 interacting with the β-face of the steroid; an interaction which is disrupted in the wildtype (FIG. 10). Additionally, the distance of the C3 ketone of the steroid towards the hydroxyl group of Tyr55 and C4N of NADPH is lower in the truncated type; implying an increased chance for the initiation of catalysis (FIG. 11). FIG. 10 shows the conformation of 5α-DHT into the binding pocket of AKR1C2. The wildtype protein is highlighted in light gray; the steroid is highlighted in black; and the main residues contributing to the binding are highlighted in dark gray. The steroid is sandwiched between Val54 and Trp227 in the truncated type. Notable is the difference in conformation of the Trp227 side chain, which interacts with the β-face of 5α-DHT, as it is flipped away in the wildtype. Trp227 is one of the main residues that hold the steroid in place.

FIG. 11 shows the distance between C3 of 5α-DHT and C4N of NADPH (A) and the distance between C3 of 5α-DHT and the hydroxyl group of Tyr55 (B). Both distances tend to be smaller in the truncated type than the wildtype.

Additionally, the effect of variants located in regulatory regions, namely rs28571848 (chr10:5019979) and rs34477787 (chr10:5071991) to overexpression of AKR1C2 and fat accumulation is already reported in [Ostinelli et al., 2021].

Using the GWAS catalog database, the inventors gathered variants present in subjects affected by obesity. The majority of them were located upstream AKR1C2 (Table 13) in proximity to the regions reported in [Ostinelli et al., 2021], namely the binding sites of retinoid acid-related orphan receptor and the glucocorticoid receptor, two transcription factors important to the regulation of AKR1C2 expression in adipose tissue.

Since lipedema is still difficult to be diagnosed, and is often misdiagnosed as obesity, the inventors discovered that these variants yield important insight in developing the link between the relative expression of AKR1C genes (in particular AKR1C2 overexpression) and lipedema. At this regard, table 13 lists variants extracted from obesity patients in the GWAS catalog database.

TABLE 13
Variants extracted from obesity patients in the GWAS catalog database

| rsID | Position | Database | Location | Frequency |
|---|---|---|---|---|
| rs145611933 | 2kb upstream | GWAS catalog | chr10:5019786 | 0.039604 |
| | AKR1C2 | | | |
| rs6601888 | 0.5kB | GWAS catalog | chr10:4983447 | 0.462373 |
| | downstream | | | |
| | AKR1C1 | | | |
| rs36032941 | 2kB upstream | GWAS catalog | chr10:5020560 | 0.233337 |
| | AKR1C2 | | | |
| rs61856103 | 2kB upstream | GWAS catalog | chr10:5019349 | 0.02690 |
| | AKR1C2 | | | |
| rs4881378 | 4kB upstream | GWAS catalog | chr10:5022148 | 0.389066 |
| | AKR1C2 | | | |
| rs61856128 | 2kB upstream | GWAS catalog | chr10:5021190 | 0.279082 |
| | AKR1C2 | | | |
| rs10795227 | 6kB upstream | GWAS catalog | chr10:5025968 | 0.223133 |
| | AKR1C2 | | | |

All the variants reported in Table 13 are located in regulatory regions of AKR1C1, AKR1C2, and AKR1C3 enzymes.

The following paragraphs deal with therapeutics for lipedema individuated by the inventors. A comprehensive list of polyphenols was submitted to molecular docking by using Autodock Vina 1.2.2 [Eberhardt et al., 2021], with AKR1C1/AKR1C2 and NADPH coordinates from PDB entry 1MRQ [Couture et al., 2003]. From molecular dynamics simulations, W227, L54, Y24 contributed to more than 50% of the overall binding energy, implying their importance in substrate specificity; thus, they were set as flexible residues during docking. The box was located at the active site, centered at x=4.29, y=33.9, z=17.06 with dimensions x=17.39, y=11.16, z=12.67 enclosing the residues that are responsible for the binding of steroids. The docking results are reported as binding affinity in Kcal/mol (the lower, the better).

Two databases were used to select potential inhibitors, namely off-label molecules (db1) and a wide range of polyphenols and other molecules (db2). From the list of the inhibitors triazolam and alprazolam seem to be the most interesting due to their specific binding capabilities to AKR1C2 and not AKR1C1. Both scored higher than the natural substrate of AKR1C2 (DHT). However, among the natural molecules, Flavanones are among the best binders to AKR1C2, although still scoring less than its natural substrate (DHT), while flavones are the best binders to AKR1C1. Flavones and flavanones differ from which other in that the latter have the C ring saturated, and unlike flavones, the double bond between positions 2 and 3 is saturated.

Molecules that inhibit AKR1C2 are of great interest, especially after evidence that AKR1C2 overexpression may be a feature of lipedema. Nonetheless, achieving specificity in inhibiting AKR1C2 without affecting the activity of AKR1C1 will be difficult, due to the similarity of these enzymes' active sites, with the main driving force of the interaction being the stacking interaction of the B ring with Y24 and W227, while L54 and V54 in AKR1C1 and AKR1C2 respectively, are responsible for substrate specificity. In any case, it is a plausible approach to use molecules that are only specific for AKR1C2 but not for AKR1C1 in the lipedema therapy and/or that exhibit a high negative binding affinity that is more negative than the binding affinity of DHT.

Finally, while there are differences in preferences towards specific molecules, it can be seen that the whole class of polyphenols binds with similar affinities to the enzyme's natural substrate, acting as competitive inhibitors. This, once again, demonstrates the ability of polyphenols to act as natural remedies and to treat lipedema.

Tables 14 and 15 show the results for AKR1C2 docking and AKR1C1 docking, respectively.

TABLE 14
AKR1C2 docking

| | Binding Affinity | | |
|---|---|---|---|
| Molecule | (kcal/mol) | Database | Class |
| Triazolam | −15.665 | Db1 | Drug |
| Alprazolam | −15.585 | Db1 | Drug |
| Canrenone | −15.376 | Db2 | Drug |
| DHT | −15.13 | | Hormone (natural |
| | | | substrate) |
| Cyanoketone | −15.011 | Db2 | Drug |
| Azastene | −14.998 | Db2 | Drug |
| 11-Ketoprogesterone | −14.989 | Db2 | Drug |
| Sophoraflavanone B | −14.901 | Db2 | Polyphenol |
| Dydrogesterone | −14.749 | Db2 | Hormone |
| Danazol | −14.571 | Db2 | Drug |
| hPGS | −14.554 | | Hormone |
| Flavanone | −14.512 | Db2 | Polyphenol |
| Diazepam | −14.427 | Db1 | Drug |
| Abietic acid | −14.4 | Db2 | Organic compound |
| Androsta-1,4,6-triene- | −14.363 | Db2 | Hormone |
| 3,17-dione | | | |
| Carbamazepine | −14.322 | Db1 | Drug |
| Flavone | −14.207 | Db2 | Polyphenol |
| Coumestrol | −14.048 | Db2 | Polyphenol |
| Isoxanthohumol | −13.98 | Db2 | Polyphenol |
| Equilin | −13.95 | Db2 | Hormone |
| Glycyrrhetinic Acid | −13.913 | Db2 | Drugs |
| Delorazepam | −13.909 | Db1 | Drug |
| 3-hydroxyflavone | −13.889 | Db2 | Polyphenol |
| 5-hydroxyflavone | −13.585 | Db2 | Polyphenol |
| Paliperidone | −13.571 | Db1 | Drug |
| Oxcarbazepine | −13.554 | Db1 | Drug |
| Daidzein | −13.485 | Db2 | Polyphenol |
| Genistein | −13.367 | Db2 | Polyphenol |
| Cinacalcet | −13.359 | Db1 | Drug |
| Mirtazapine | −13.316 | Db1 | Drug |
| 3-7-dihydroxyflavone | −13.287 | Db2 | Polyphenol |
| Bicalutamide | −13.263 | Db1 | Drug |
| Risperidone | −13.179 | Db1 | Drug |
| Phenytoin | −13.115 | Db1 | Drug |
| Pallidol | −13.11 | Db2 | Polyphenol |
| Ursodeoxycholic acid | −13.044 | Db1 | Drug |
| 6-Prenylnaringenin | −13.016 | Db2 | Polyphenol |
| Canaglifozin | −12.374 | | |
| Tauroursodeoxycholic | −11.903 | Db1 | Drug |
| acid | | | |
| Empaglifozin | −11.318 | Db1 | Drug |
| Dapaglifozin | −10.877 | Db1 | Drug |

TABLE 15
AKR1C1 docking

| | Binding | | |
|---|---|---|---|
| | Affinity | | |
| Molecule | (kcal/mol) | Database | Class |
| Flavone | −17.215 | Db2 | Polyphenol |
| Flavanone | −16.755 | Db2 | Polyphenol |
| 3-Hydroxyflavone | −16.468 | Db2 | Polyphenol |
| Equilin | −16.435 | Db2 | Hormone |
| Coumestrol | −16.286 | Db2 | Polyphenol |
| hPGS | −16.184 | | Hormone (natural |
| | | | substrate) |
| 5-hydroxyflavone | −16.105 | Db2 | Polyphenol |
| Abietic acid | −15.934 | Db2 | Organic compound |
| Mirtazapine | −15.591 | Db1 | Drug |
| Bromazepam | −15.565 | Db1 | Drug |
| 3-7-dihydroxyflavone | −15.495 | Db2 | Polyphenol |
| 7-hydroxyflavone | −15.448 | Db2 | Polyphenol |
| Sesamin | −15.387 | Db2 | Lignan |
| Lorazepam | −15.355 | Db1 | Drug |
| Rhoifolin | −15.318 | Db2 | Polyphenol |
| Pinocembrin | −15.298 | Db2 | Polyphenol |
| 5-7-dyhydroxyflavone | −15.274 | Db2 | Polyphenol |
| Chrysin | −15.274 | Db2 | Polyphenol |
| Coumarin | −15.267 | Db2 | Organic compound |
| Canrenone | −15.26 | Db2 | Drug |
| Oxcarbazepine | −15.191 | Db1 | Drug |
| Baicalein | −15.161 | Db2 | Polyphenol |
| Naringenin | −15.149 | Db2 | Polyphenol |
| Risperidone | −15.146 | Db1 | Drug |
| Phenytoin | −15.132 | Db1 | Drug |
| Pallidol | −15.036 | Db2 | Polyphenol |
| Sesamolin | −15.013 | Db2 | Lignan |
| Alprazolam | −14.983 | Db1 | Drug |
| 7,3′,4′- | −14.973 | Db2 | Polyphenol |
| Trihydroxyflavone | | | |
| Azastene | −14.925 | Db2 | Drug |
| Luteolin | −14.922 | Db2 | Polyphenol |
| Scutellarein | −14.921 | Db2 | Polyphenol |
| 11-Ketoprogesterone | −14.517 | Db2 | Drug |
| Cyanoketone | −14.434 | Db2 | Drug |
| Androsta-1,4,6-triene- | −14.088 | Db2 | Drug |
| 3,17-dione | | | |
| Danazol | −13.931 | Db2 | Drug |

The screening of inhibitors allowed to individuate sets of drugs and natural molecules that are specific to the inhibition of AKR1C2 but not AKR1C1. In addition, Random Accelerated Molecular Dynamics (RAMD) [Kokh et al. 2018] can be used to study the residence time of the ligands bound to the enzyme in order to understand variants in AKR1C2 that create an enzyme with increased binding capabilities to its natural substrate. At the same time, the results can be extended on the binding affinity of different natural inhibitors to AKR1C2, for accurate predictions on the molecules that inhibit AKR1C2.

The current data have opened the way to establish a more rigorous correlation between these genes and the pathology. Having said that, it is useful to have a method that screens variants in these genes that is disruptive to the protein's function. Therefore, the inventors developed a criterion to quickly predict disruptive missense variants found in AKR1C1 or AKR1C2 (for the latter, the interest goes to variants that are excluded by the inventors' criterion, since they expect an increased activity of AKR1C2 to lead to lipedema). The criterion is applied on a landscape of these variants derived from their properties as explained in the next paragraph (an example of this landscape for AKR1C2 can be found in Table 17).

The criterion developed by the inventors consisted in the evaluation of the following properties: the position's entropies, the predicted ΔΔG of the variant, and the positions' contribution to the overall substrate binding energy or to catalysis. The variants in AKR1C2 that concern residues with high contribution to the overall substrate binding energy with respect to the natural compounds (natural binding partners) or that contribute to catalysis are favored, and are excluded from the list of the pathogenic variants linked to lipedema (e.g. Leu54, Tyr24, and Trp227 which account for roughly 50% of the total binding energy of AKR1C1 to Progesterone, or Tyr55, Asp50, Lys84, His117 which form the catalytic tetrad); then the entropy (conservation of a position) and the predicted ΔΔG (predicted change of the protein's fold stability when the variant is introduced) are taken into account. If the variant is located in a position with relative entropy >5 OR predicted ΔΔG<−3, it is considered it as potentially disruptive, and subject it to further studies. This way, by including both criteria, the criterion accounts for the importance of the position itself (entropy), and for the changes in physicochemical properties caused by the substitution of one residue with another (predicted ΔΔG).

It must be restated that one of the leading factors to lipedema is hormone imbalance, which is highly sensitive to environmental factors, and genetic factors alone could not lead to the onset of this pathology. Having a selection of disruptive variants in genes that are correlated to lipedema, helps in the early detection of individual's predisposition to lipedema, as well as detect that lipedema is indeed more common than previously thought and could be present in higher frequencies in the general population (considering that it is often misdiagnosed or not diagnosed at all). The inventors ran these predictions on a set of AKR1C1 and AKR1C2 variants in the general population, extracted from the GnomAD database. In Table 16 an example of the disruptive variants in AKR1C1 filtered according to the inventors' criterion is presented, and in Table 17 the whole unfiltered predictions of AKR1C2 variants are presented.

TABLE 16
Evaluation of AKR1C1 variants in the general population according to the inventors' criteria.

| | | | Predicted | | |
|---|---|---|---|---|---|
| | Amino Acid | Relative | ΔΔG | | Allele |
| rsID | Variant | Entropy | (kcal/mol) | Outcome | Frequency |
| rs999611958 | His48Arg | 5.32 | −1.52 | Destabilizing | 7.95e−06 |
| | | | | (Disrupts folding) | |
| — | Tyr55His | 5.00 | −1 | Destabilizing | 3.97e−06 |
| | | | | (Disrupts | |
| | | | | catalysis) | |
| rs1462840208 | Trp86Ser | 6.07 | −3.769 | Highly | 4.03e−06 |
| | | | | Destabilizing | |
| | | | | (Disrupts folding) | |
| rs754792432 | His117Asp | 5.32 | −2.164 | Highly | 3.98e−06 |
| | | | | Destabilizing | |
| | | | | (Disrupts | |
| | | | | catalysis) | |
| rs778903438 | His117Pro | 5.32 | −1.26 | Destabilizing | 2.83e−05 |
| | | | | (Disrupts | |
| | | | | catalysis) | |
| — | His117Arg | 5.32 | −1.112 | Destabilizing | 6.57e−06 |
| | | | | (Disrupts | |
| | | | | catalysis) | |
| rs752532298 | Pro119Thr | 4.53 | −2.639 | Highly | 3.98e−06 |
| | | | | Destabilizing | |
| | | | | (Disrupts folding) | |
| rs782186892 | Trp227Arg | 5.83 | −0.779 | Destabilizing | 1.59e−05 |
| | | | | (Disrupts substrate | |
| | | | | binding) | |

TABLE 17
Predictions (landscape) of AKR1C2 variants in the general population

| WILD _ | residue_ | | | | |
|---|---|---|---|---|---|
| RES | num | MUT_RES | RSA | PRED_DDG | rel_entropy |
| L | 10 | P | 0.3 | −1.709 | 3.38669 |
| N | 11 | S | 14.9 | −0.721 | 4.128421 |
| H | 14 | P | 46.3 | −0.105 | 4.510611 |
| F | 15 | L | 60.3 | −0.997 | 2.732814 |
| F | 15 | V | 60.3 | −1.257 | 2.732814 |
| V | 18 | I | 10.4 | −0.677 | 2.866382 |
| G | 20 | E | 0 | −1.964 | 3.476417 |
| T | 23 | P | 0 | −0.611 | 3.780331 |
| A | 25 | T | 8.2 | −1.16 | 2.513344 |
| A | 25 | G | 8.2 | −1.243 | 2.513344 |
| A | 25 | V | 8.2 | −0.069 | 2.513344 |
| V | 29 | I | 34.8 | −0.547 | 2.649598 |
| V | 29 | A | 34.8 | −1.121 | 2.649598 |
| P | 30 | L | 64.1 | −0.274 | 4.064559 |
| K | 33 | E | 60.6 | −0.003 | 2.274512 |
| L | 35 | S | 36.9 | −1.982 | 0.835598 |
| A | 37 | S | 1.5 | −1.354 | 2.335156 |
| A | 37 | T | 1.5 | −0.993 | 2.335156 |
| K | 39 | R | 26.3 | −1.125 | 3.513024 |
| L | 40 | W | 16.2 | −1.947 | 1.569845 |
| I | 42 | T | 4.6 | −3.2 | 3.757671 |
| E | 43 | K | 46.7 | −0.019 | 2.896437 |
| E | 43 | V | 46.7 | −0.187 | 2.896437 |
| A | 44 | D | 0.3 | −1.477 | 2.389812 |
| F | 46 | L | 0.7 | −1.849 | 3.820853 |
| H | 48 | R | 1.6 | −1.846 | 5.321928 |
| I | 49 | T | 0 | −2.778 | 3.361971 |
| A | 52 | S | 0 | −1.823 | 3.447281 |
| V | 61 | I | 0 | −0.794 | 3.147864 |
| G | 62 | R | 0 | −1.359 | 3.590745 |
| A | 64 | V | 0 | −0.546 | 3.543943 |
| I | 65 | V | 0 | −1.664 | 3.827464 |
| A | 70 | T | 85.8 | −0.65 | 2.796526 |
| A | 70 | E | 85.8 | −0.834 | 2.796526 |
| G | 72 | C | 97.6 | −0.913 | 3.590745 |
| S | 73 | R | 51.6 | −0.161 | 2.677572 |
| E | 77 | V | 79.1 | −0.096 | 3.737572 |
| E | 77 | G | 79.1 | −0.664 | 3.737572 |
| D | 78 | G | 40.3 | −0.892 | 3.770501 |
| I | 79 | L | 0 | −1.715 | 3.034017 |
| I | 79 | K | 0 | −2.783 | 3.034017 |
| I | 79 | M | 0 | −1.854 | 3.034017 |
| F | 80 | S | 7.3 | −3.106 | 4.433296 |
| R | 91 | G | 39.2 | −1.35 | 3.179009 |
| R | 91 | L | 39.2 | −0.067 | 3.179009 |
| R | 91 | Q | 39.2 | −0.648 | 3.179009 |
| P | 92 | T | 38.9 | −1.322 | 4.183596 |
| P | 92 | R | 38.9 | −0.544 | 4.183596 |
| P | 97 | S | 51.4 | −1.588 | 3.088188 |
| S | 102 | T | 0.2 | −0.984 | 3.794215 |
| K | 104 | I | 87.4 | 0.43 | 3.371506 |
| L | 108 | F | 6.3 | −1.422 | 3.150925 |
| Y | 110 | F | 23.9 | −1.026 | 4.720751 |
| D | 112 | V | 12.9 | −0.522 | 4.265345 |
| D | 112 | E | 12.9 | −0.839 | 4.265345 |
| L | 113 | V | 2 | −1.891 | 3.442222 |
| H | 117 | D | 12.8 | −1.88 | 5.321928 |
| H | 117 | R | 12.8 | −0.873 | 5.321928 |
| H | 117 | P | 12.8 | −1.082 | 5.321928 |
| V | 128 | L | 41.8 | −0.498 | 1.50576 |
| V | 128 | M | 41.8 | −0.396 | 1.50576 |
| P | 130 | L | 11.3 | −0.745 | 4.30391 |
| K | 131 | I | 49.6 | 0.405 | 1.98148 |
| K | 131 | R | 49.6 | −0.508 | 1.98148 |
| D | 132 | E | 42.9 | −0.444 | 3.557757 |
| N | 134 | S | 89.4 | 0.001 | 2.637745 |
| T | 141 | I | 109.6 | −0.17 | 2.277381 |
| V | 142 | A | 7.7 | −1.424 | 3.488624 |
| C | 145 | F | 22 | −1.442 | 4.460404 |
| T | 147 | I | 0 | 0.149 | 3.843052 |
| A | 150 | P | 16 | −0.626 | 3.269635 |
| C | 154 | R | 0.4 | −1.542 | 4.998057 |
| A | 157 | E | 32 | −0.768 | 3.148574 |
| A | 160 | P | 0 | −0.575 | 2.458647 |
| K | 16 | R | 39.1 | −0.837 | 3.845507 |
| I | 163 | M | 0.9 | −1.509 | 4.011588 |
| G | 164 | R | 0 | −1.067 | 3.590745 |
| V | 165 | L | 0 | −0.364 | 3.667674 |
| S | 166 | T | 1.4 | −1.11 | 4.024426 |
| N | 169 | S | 39.1 | −0.847 | 4.608232 |
| L | 177 | P | 8.1 | −1.654 | 3.38466 |
| G | 181 | V | 127.5 | −0.456 | 3.373976 |
| K | 185 | Q | 38.3 | −1.014 | 4.038387 |
| N | 189 | K | 0 | −0.703 | 4.465996 |
| D | 204 | Y | 61.7 | −0.437 | 2.873165 |
| D | 204 | G | 61.7 | −0.984 | 2.873165 |
| Y | 196 | H | 3.5 | −2.644 | 4.609254 |
| F | 197 | S | 18.1 | −2.591 | 2.554424 |
| N | 198 | K | 8.3 | −0.337 | 4.470952 |
| K | 201 | T | 107.2 | −0.172 | 3.950785 |
| C | 206 | R | 0 | −1.547 | 5.380822 |
| K | 209 | E | 40.3 | 0.006 | 3.222591 |
| I | 211 | V | 0 | −1.864 | 3.940261 |
| L | 213 | Q | 0 | −2.664 | 3.212573 |
| Y | 216 | N | 17.3 | −1.532 | 4.086959 |
| Y | 216 | C | 17.3 | −0.857 | 4.086959 |
| S | 217 | G | 17 | −1.071 | 2.890275 |
| S | 217 | T | 17 | −0.577 | 2.890275 |
| S | 221 | T | 6.3 | −0.712 | 3.240127 |
| S | 221 | C | 6.3 | −0.399 | 3.240127 |
| S | 221 | Y | 6.3 | −0.358 | 3.240127 |
| R | 223 | Q | 24.4 | −0.63 | 3.792231 |
| P | 230 | R | 48.3 | 0.165 | 2.710676 |
| P | 233 | L | 45.9 | −0.483 | 4.466187 |
| P | 233 | R | 45.9 | −0.608 | 4.466187 |
| D | 238 | G | 4.6 | −0.162 | 3.391944 |
| D | 238 | E | 4.6 | −0.571 | 3.391944 |
| V | 240 | F | 47.3 | −1.099 | 3.0717 |
| C | 242 | Y | 44.5 | −1.1 | 2.963712 |
| L | 244 | S | 10.3 | −3.072 | 2.046985 |
| A | 245 | T | 6 | −1.947 | 3.608868 |
| K | 246 | E | 94.8 | 0.024 | 3.548748 |
| K | 247 | N | 67.6 | 0.033 | 3.858181 |
| K | 249 | E | 44.7 | −0.011 | 2.6647 |
| R | 250 | Q | 21.6 | −0.869 | 3.295204 |
| T | 251 | I | 38.5 | −0.108 | 3.127317 |
| P | 252 | A | 24.6 | −1.899 | 4.073423 |
| A | 253 | T | 15.4 | −1.625 | 2.975594 |
| L | 254 | V | 0.5 | −1.916 | 2.694096 |
| A | 256 | S | 0 | −1.843 | 3.42224 |
| R | 258 | C | 13.4 | −1.983 | 4.222077 |
| R | 258 | H | 13.4 | −2.394 | 4.222077 |
| Y | 259 | C | 0 | −2.141 | 4.232601 |
| Q | 262 | H | 25 | −0.89 | 4.615449 |
| R | 263 | C | 6.1 | −1.347 | 4.050349 |
| R | 263 | L | 6.1 | −0.346 | 4.050349 |
| R | 263 | H | 6.1 | −1.862 | 4.050349 |
| V | 266 | A | 0.9 | −2.23 | 3.677163 |
| L | 268 | R | 6.3 | −1.095 | 3.32942 |
| S | 271 | N | 14.3 | −0.612 | 3.950894 |
| S | 271 | R | 14.3 | −0.438 | 3.950894 |
| Y | 272 | N | 13.2 | −1.62 | 3.282352 |
| N | 273 | S | 50.8 | −0.346 | 2.587955 |
| R | 276 | C | 63.7 | −0.897 | 3.649566 |
| R | 276 | H | 63.7 | −1.418 | 3.649566 |
| N | 280 | K | 3.6 | −0.393 | 4.385006 |
| M | 293 | T | 18.2 | −1.837 | 5.103661 |
| K | 294 | Q | 74.6 | 0.05 | 3.098772 |
| A | 295 | T | 57 | −1.01 | 1.69993 |
| T | 307 | A | 47.6 | −0.832 | 1.044365 |
| D | 309 | H | 68.6 | 0.703 | 1.339211 |
| I | 310 | T | 41.9 | −1.697 | 1.359858 |
| G | 313 | R | 111.7 | −0.374 | 1.681438 |
| P | 315 | L | 94.1 | −0.299 | 4.042938 |
| N | 316 | T | 29.6 | −0.574 | 2.108382 |

### REFERENCES

Abraham M J, Murtola T, Schulz R, Pall S, Smith J C, Hess B, and Lindahl E. GROMACS: High performance molecular simulations through multi-level parallelism from laptops to supercomputers. SoftwareX. 2015;1-2:19-25. doi.org/10.1016/j.softx.2015.06.001.
Bauer A T, von Lukowicz D, Lossagk K, Aitzetmueller M, Moog P, Cerny M, Erne H, Schmauss D, Duscher D, Machens H G. New Insights on Lipedema: The Enigmatic Disease of the Peripheral Fat. Plast Reconstr Surg. 2019 December; 144(6):1475-1484. doi: 10.1097/PRS.0000000000006280. PMID: 31764671.
Blanchette S, Blouin K, Richard C, Dupont P, Luu-The V, Tchernof A. Expression and activity of 20alpha-hydroxysteroid dehydrogenase (AKR1C1) in abdominal subcutaneous and omental adipose tissue in women. J Clin Endocrinol Metab. 2005 January; 90(1):264-70. doi: 10.1210/jc.2004-0583. Epub 2004 Oct. 19. PMID: 15494462.
Blouin K, Richard C, Brochu G, Hould F S, Lebel S, Marceau S, Biron S, Luu-The V, Tchernof A. Androgen inactivation and steroid-converting enzyme expression in abdominal adipose tissue in men. J Endocrinol. 2006 December; 191(3):637-49. doi: 10.1677/joe.1.06365. PMID: 17170221.
Blouin K, Nadeau M, Mailloux J, Daris M, Lebel S, Luu-The V, T. A. Pathways of adipose tissue androgen metabolism in women: depot differences and modulation by adipogenesis. Am. J. Physiol. Endocrinol. Metab. 2009, 296, doi:10.1152/AJPEND0.00039.2008.
Brozic P, Cesar J, Kovac A, Davies M, Johnson A P, Fishwick C W, Lanisnik Rizner T, Gobec S. Derivatives of pyrimidine, phthalimide and anthranilic acid as inhibitors of human hydroxysteroid dehydrogenase AKR1C1. Chem Biol Interact. 2009 Mar. 16; 178(1-3):158-64. doi: 10.1016/j.cbi.2008.10.019. Epub 2008 Oct. 22. PMID: 19007763.
Buck D W 2nd, Herbst KL. Lipedema: A Relatively Common Disease with Extremely Common Misconceptions. Plast Reconstr Surg Glob Open. 2016 Sep. 28; 4(9):e1043. doi: 10.1097/GOX.0000000000001043. PMID: 27757353; PMCID: PMC5055019.
Buso G, Depairon M, Tomson D, Raffoul W, Vettor R, Mazzolai L. Lipedema: A Call to Action! Obesity (Silver Spring). 2019 October; 27(10):1567-1576. doi: 10.1002/oby.22597. PMID: 31544340; PMCID: PMC6790573.
Byrns M C, Duan L, Lee S H, Blair I A, Penning T M. Aldo-keto reductase 1C3 expression in MCF-7 cells reveals roles in steroid hormone and prostaglandin metabolism that may explain its over-expression in breast cancer. J Steroid Biochem Mol Biol. 2010 Feb. 15; 118(3):177-87. doi: 10.1016/j.jsbmb.2009.12.009. Epub 2009 Dec. 28. PMID: 20036328; PMCID: PMC2819162.
Byrns M C, Mindnich R, Duan L, Penning T M. Overexpression of aldo-keto reductase 1C3 (AKR1C3) in LNCaP cells diverts androgen metabolism towards testosterone resulting in resistance to the 5α-reductase inhibitor finasteride. J Steroid Biochem Mol Biol. 2012 May; 130(1-2):7-15. doi: 10.1016/j.jsbmb.2011.12.012. Epub 2012 Jan. 12. PMID: 22265960; PMCID: PMC3319280.
Couture J F, Legrand P, Cantin L, Luu-The V, Labrie F, Breton R. Human 20alpha-hydroxysteroid dehydrogenase: crystallographic and site-directed mutagenesis studies lead to the identification of an alternative binding site for C21-steroids. J Mol Biol. 2003; 331(3):593-604. doi:10.1016/s0022-2836(03)00762-9.
Dhayat N A, Marti N, Kollmann Z, Troendle A, Bally L, Escher G, Grossl M, Ackermann D, Ponte B, Pruijm M, Muller M, Vogt B, Birkhauser M H, Bochud M, Fltick C E; members of the SKIPOGH Study Group. Urinary steroid profiling in women hints at a diagnostic signature of the polycystic ovary syndrome: A pilot study considering neglected steroid metabolites. PLoS One. 2018 Oct. 11; 13(10):e0203903. doi: 10.1371/journal.pone.0203903. PMID: 30308019; PMCID: PMC6181287.
Di Renzo L, Cinelli G, Romano L, Zomparelli S, Lou De Santis G, Nocerino P, Bigioni G, Arsini L, Cenname G, Pujia A, Chiricolo G, De Lorenzo A. Potential Effects of a Modified Mediterranean Diet on Body Composition in Lipoedema. Nutrients. 2021 Jan. 25; 13(2):358. doi: 10.3390/nu13020358. PMID: 33504026.
Dozier B L, Watanabe K, Duffy D M. Two pathways for prostaglandin F2 alpha synthesis by the primate periovulatory follicle. Reproduction. 2008 July; 136(1):53-63. doi: 10.1530/REP-07-0514. Epub 2008 Apr. 4. PMID: 18390687; PMCID: PMC2656351.
Eberhardt J, Santos-Martins D, Tillack A F, Forli S. AutoDock Vina 1.2.0: New Docking Methods, Expanded Force Field, and Python Bindings. J Chem Inf Model. 2021 Aug. 23; 61(8):3891-3898. doi: 10.1021/acs.jcim.1c00203. Epub 2021 Jul. 19. PMID: 34278794.
Fife C E, Maus E A, Carter M J. Lipedema: a frequently misdiagnosed and misunderstood fatty deposition syndrome. Adv Skin Wound Care. 2010 February; 23(2):81-92; quiz 93-4. doi: 10.1097/01.ASW.0000363503.92360.91. PMID: 20087075.
Fong P, Tong H H, Ng K H, Lao C K, Chong C I, Chao C M. In silico prediction of prostaglandin D2 synthase inhibitors from herbal constituents for the treatment of hair loss. J Ethnopharmacol. 2015 Dec. 4; 175:470-80. doi: 10.1016/j.jep.2015.10.005. Epub 2015 Oct. 9. PMID: 26456343.
Forner-Cordero, I.; Szolnoky, G.; Forner-Cordero, A.; Kemeny, L. Lipedema: An Overview of Its Clinical Manifestations, Diagnosis and Treatment of the Disproportional Fatty Deposition Syndrome—Systematic Review. Clin Obes 2012, 2, 86-95, https://doi.org/10.1111/j.1758-8111.2012.00045.x.
Gavin K M, Cooper E E, H.R. Estrogen receptor protein content is different in abdominal than gluteal subcutaneous adipose tissue of overweight-to-obese premenopausal women. Metabolism. 2013, 62, 1180-1188, doi:10.1016/J.METABOL.2013.02.010.
Genheden S, Ryde U. The MM/PBSA and MM/GBSA methods to estimate ligand-binding affinities. Expert Opin Drug Discov. 2015 May; 10(5):449-61. doi: 10.1517/17460441.2015.1032936. Epub 2015 Apr. 2. PMID: 25835573; PMCID: PMC4487606.
Gray J M, Wade G N. Food intake, body weight, and adiposity in female rats: actions and interactions of progestins and antiestrogens. Am J Physiol. 1981 May; 240(5):E474-81. doi: 10.1152/ajpendo.1981.240.5.E474. PMID: 7195153.
Grigoriadis, D.; Sackey, E.; Riches, K.; Zanten, M. van; Brice, G.; England, R.; Mills, M.; Dobbins, S. E.; Lipoedema Consortium, G.E.R.C.; Jeffery, S.; et al. Investigation of clinical characteristics and genome associations in the ‘UK Lipoedema’ cohort. medRxiv 2021, 2021.06.15.21258988, doi:10.1101/2021.06.15.21258988.
Haidari F, Homayouni F, Helli B, Haghighizadeh M H, Farahmandpour F. Effect of chlorella supplementation on systematic symptoms and serum levels of prostaglandins, inflammatory and oxidative markers in women with primary dysmenorrhea. Eur J Obstet Gynecol Reprod Biol. 2018 October; 229:185-189. doi: 10.1016/j.ejogrb.2018.08.578. Epub 2018 Aug. 27. PMID: 30205315.
Hara A, Matsuura K, Tamada Y, Sato K, Miyabe Y, Deyashiki Y, Ishida N. Relationship of human liver dihydrodiol dehydrogenases to hepatic bile-acid-binding protein and an oxidoreductase of human colon cells. Biochem J. 1996 Jan. 15; 313 (Pt 2)(Pt 2):373-6. doi: 10.1042/bj3130373. PMID: 8573067; PMCID: PMC1216918.
Helmersson J, Arnlov J, Vessby B, Larsson A, Alfthan G, Basu S. Serum selenium predicts levels of F2-isoprostanes and prostaglandin F2alpha in a 27 year follow-up study of Swedish men. Free Radic Res. 2005 July; 39(7):763-70. doi: 10.1080/10715760500108513. PMID: 16036356.
Jia X J, Liu L X, Tian Y M, Wang R, Lu Q. The correlation between oxidative stress level and intra-abdominal fat in obese males. Medicine (Baltimore). 2019 February; 98(7):e14469. doi: 10.1097/MD.0000000000014469. PMID: 30762765; PMCID: PMC6408049.
Katzer K, Hill J L, McIver K B, Foster M T. Lipedema and the Potential Role of Estrogen in Excessive Adipose Tissue Accumulation. Int J Mol Sci. 2021 Oct. 29; 22(21):11720.
Kiani A K, Mor M, Bernini A, Fulcheri E, Michelini S, Herbst K L, Buffelli F, Belgrado J P, Kaftalli J, Stuppia L, Dautaj A, Dhuli K, Guda T, Manara E, Maltese P E, Michelini S, Chiurazzi P, Paolacci S, Ceccarini M R, Beccari T, Bertelli M. Steroid-converting enzymes in human adipose tissues and fat deposition with a focus on AKR1C enzymes. Eur Rev Med Pharmacol Sci. 2021 December; 25(1 Suppl):23-32. doi: 10.26355/eurrev_202112_27330. PMID: 34890031.
Kim J B, Spiegelman B M. ADD1/SREBP1 promotes adipocyte differentiation and gene expression linked to fatty acid metabolism. Genes Dev. 1996 May 1; 10(9):1096-107. doi: 10.1101/gad.10.9.1096. PMID: 8654925.
Koeberle A, Bauer J, Verhoff M, Hoffmann M, Northoff H, Werz O. Green tea epigallocatechin-3-gallate inhibits microsomal prostaglandin E(2) synthase-1. Biochem Biophys Res Commun. 2009 Oct. 16; 388(2):350-4. doi: 10.1016/j.bbrc.2009.08.005. Epub 2009 Aug. 6. PMID: 19665000.
Kokh D B, Amaral M, Bomke J, Gradler U, Musil D, Buchstaller H P, Dreyer M K, Frech M, Lowinski M, Vallee F, Bianciotto M, Rak A, Wade R C. Estimation of Drug-Target Residence Times by τ-Random Acceleration Molecular Dynamics Simulations. J Chem Theory Comput. 2018 Jul. 10; 14(7):3859-3869. doi: 10.1021/acs.jctc.8b00230. Epub 2018 Jun. 4. PMID: 29768913.
Kruppa P, Georgiou I, Biermann N, Prantl L, Klein-Weigel P, Ghods M. Lipedema-Pathogenesis, Diagnosis, and Treatment Options. Dtsch Arztebl Int. 2020 Jun. 1; 117(22-23):396-403. doi: 10.3238/arzteb1.2020.0396. PMID: 32762835; PMCID: PMC7465366.
Lacasa D, Le Liepvre X, Ferre P, Dugail I. Progesterone stimulates adipocyte determination and differentiation 1/sterol regulatory element-binding protein lc gene expression. potential mechanism for the lipogenic effect of progesterone in adipose tissue. J Biol Chem. 2001 Apr. 13; 276(15):11512-6. doi: 10.1074/jbc.M008556200. Epub 2001 Jan. 16. PMID: 11278421.
Lepak N M, Serrero G. Prostaglandin F2 alpha stimulates transforming growth factor-alpha expression in adipocyte precursors. Endocrinology. 1995 August; 136(8):3222-9. doi: 10.1210/endo.136.8.7628355. PMID: 7628355.
Matsuura K, Deyashiki Y, Sato K, Ishida N, Miwa G, Hara A. Identification of amino acid residues responsible for differences in substrate specificity and inhibitor sensitivity between two human liver dihydrodiol dehydrogenase isoenzymes by site-directed mutagenesis. Biochem J. 1997 Apr. 1; 323 (Pt 1)(Pt 1):61-4. doi: 10.1042/bj3230061. PMID: 9173902; PMCID: PMC1218315.
Michelini S, Chiurazzi P, Marino V, Dell'Orco D, Manara E, Baglivo M, Fiorentino A, Maltese P E, Pinelli M, Herbst K L, Dautaj A, Bertelli M. Aldo-Keto Reductase 1C1 (AKR1C1) as the First Mutated Gene in a Family with Nonsyndromic Primary Lipedema. Int J Mol Sci. 2020 Aug. 29; 21(17):6264. doi: 10.3390/ijms21176264. PMID: 32872468; PMCID: PMC7503355.
Michelini, S.; Herbst, K. L.; Precone, V.; Manara, E.; Marceddu, G.; Dautaj, A.; Maltese, P. E.; Paolacci, S.; Ceccarini, M. R.; Beccari, T.; et al. A Multi-Gene Panel to Identify Lipedema-Predisposing Genetic Variants by a Next-Generation Sequencing Strategy. J Pers Med 2022, 12, 268, https://doi.org/10.3390/jpm12020268.
Miller C W, Casimir D A, Ntambi J M. The mechanism of inhibition of 3T3-L1 preadipocyte differentiation by prostaglandin F2alpha. Endocrinology. 1996 December; 137(12):5641-50. doi: 10.1210/endo.137.12.8940395. PMID: 8940395.
Milne G L, Dai Q, Roberts L J 2nd. The isoprostanes—25 years later. Biochim Biophys Acta. 2015 April; 1851(4):433-45. doi: 10.1016/j.bbalip.2014.10.007. Epub 2014 Oct 30. PMID: 25449649; PMCID: PMC5404383.
Ostinelli G, Vijay J, Vohl M C, Grundberg E, Tchernof A. AKR1C2 and AKR1C3 expression in adipose tissue: Association with body fat distribution and regulatory variants. Mol Cell Endocrinol. 2021 May 1; 527:111220. doi: 10.1016/j.mce.2021.111220. Epub 2021 Mar. 3. PMID: 33675863; PMCID: PMC8052191.
Pallai R, Simpkins H, Chen J, Parekh H K. The CCAAT box binding transcription factor, nuclear factor-Y (NF-Y) regulates transcription of human aldo-keto reductase 1C1 (AKR1C1) gene. Gene. 2010 Jul, 1; 459(1-2):11-23. doi: 10.1016/j.gene.2010.03.006. Epub 2010 Mar. 23. PMID: 20338228; PMCID: PMC2874818.
Paolacci, S.; Precone, V.; Acquaviva, F.; Chiurazzi, P.; Fulcheri, E.; Pinelli, M.; Buffelli, F.; Michelini, S.; Herbst, K. L.; Unfer, V.; et al. Genetics of Lipedema: New Perspectives on Genetic Research and Molecular Diagnoses. Eur Rev Med Pharmacol Sci 2019, 23, 5581-5594, https://doi.org/10.26355/eurrev 201907 18292.
Penning T M, Wangtrakuldee P, Auchus R J. Structural and Functional Biology of Aldo-Keto Reductase Steroid-Transforming Enzymes. Endocr Rev. 2019 Apr. 1; 40(2):447-475. doi: 10.1210/er.2018-00089. PMID: 30137266; PMCID: PMC6405412.
Penning, Trevor M., Molecular Endocrinology of Hydroxysteroid Dehydrogenases, Endocrine Reviews, Volume 18, Issue 3, 1 Jun. 1997, Pages 281-305, https://doi.org/10.1210/edrv.18.3.0302.
Precone, V.; Barati, S.; Paolacci, S.; Salgarello, M.; Visconti, G.; Gentileschi, S.; Guerri, G.; Gagliardi, L.; Aquilanti, B.; Matera, G.; et al. Genetic Syndromes with Localized Subcutaneous Fat Tissue Accumulation. Acta Biomed 2019, 90, 90-92, https://doi.org/10.23750/abm.v90i10-S.8767.
Quinkler M, Buj alska I J, Tomlinson J W, Smith D M, Stewart P M. Depot-specific prostaglandin synthesis in human adipose tissue: a novel possible mechanism of adipogenesis. Gene. 2006 Oct. 1; 380(2):137-43. doi: 10.1016/j.gene.2006.05.026. Epub 2006 Jun. 10. PMID: 16842938.
Rizner T L, Lin H K, Peehl D M, Steckelbroeck S, Bauman D R, Penning T M. Human type 3 3alpha-hydroxysteroid dehydrogenase (aldo-keto reductase 1C2) and androgen metabolism in prostate cells. Endocrinology. 2003 July; 144(7):2922-32. doi: 10.1210/en.2002-0032. PMID: 12810547.
Rizner T L, Smuc T, Rupreht R, Sinkovec J, Penning T M. AKR1C1 and AKR1C3 may determine progesterone and estrogen ratios in endometrial cancer. Mol Cell Endocrinol. 2006 Mar. 27; 248(1-2):126-35. doi: 10.1016/j.mce.2005.10.009. Epub 2005 Dec. 9. PMID: 16338060.
Son M, Park C, Kwon S G, Bang W Y, Kim S W, Kim C W, Lee K W. Structural importance of the C-terminal region in pig aldo-keto reductase family 1 member C1 and their effects on enzymatic activity. BMC Struct Biol. 2015 Jan. 13; 15:1. doi: 10.1186/s12900-014-0028-7. PMID: 25583233; PMCID: PMC4310174.
Strait B J, Dewey T G. The Shannon information entropy of protein sequences. Biophys J. 1996 July; 71(1):148-55. doi: 10.1016/50006-3495(96)79210-X. PMID: 8804598; PMCID: PMC1233466.
Taketani Y, Yamagishi R, Fujishiro T, Igarashi M, Sakata R, Aihara M. Activation of the prostanoid FP receptor inhibits adipogenesis leading to deepening of the upper eyelid sulcus in prostaglandin-associated periorbitopathy. Invest Ophthalmol Vis Sci. 2014 Mar. 4; 55(3):1269-76. doi: 10.1167/iovs.13-12589. PMID: 24508785.
Tchernof A, Mansour MF, Pelletier M, Boulet M M, Nadeau M, L.-T. V Updated survey of the steroid-converting enzymes in human adipose tissues. J. Steroid Biochem. Mol. Biol. 2015, 147, 56-69, doi:10.1016/J.JSBMB.2014.11.011.
Torre Y S, Wadeea R, Rosas V, Herbst K L. Lipedema: friend and foe. Horm Mol Biol Clin Investig. 2018 Mar. 9; 33(1):/j/hmbci.2018.33.issue-1/hmbci-2017-0076/hmbci-2017-0076.xml. doi: 10.1515/hmbci-2017-0076. PMID: 29522416; PMCID: PMC5935449.
Volat F E, Pointud J C, Pastel E, Morio B, Sion B, Hamard G, Guichardant M, Colas R, Lefrançois-Martinez A M, Martinez A. Depressed levels of prostaglandin F2α in mice lacking Akr1b7 increase basal adiposity and predispose to diet-induced obesity. Diabetes. 2012 November; 61(11):2796-806. doi: 10.2337/db11-1297. Epub 2012 Jul. 30. PMID: 22851578; PMCID: PMC3478517.
Wade G N. Some effects of ovarian hormones on food intake and body weight in female rats. J Comp Physiol Psychol. 1975 January; 88(1):183-93. doi: 10.1037/h0076186. PMID: 1120795.
Warren, A. G.; Janz, B. A.; Borud, L. J.; Slavin, S. A. Evaluation and Management of the Fat Leg Syndrome. Plast Reconstr Surg 2007, 119, 9e-15e, https://doi.org/10.1097/01.prs.0000244909.82805.dc.
Zeng C, Zhu D, You J, Dong X, Yang B, Zhu H, He Q. Liquiritin, as a Natural Inhibitor of AKR1C1, Could Interfere With the Progesterone Metabolism. Front Physiol. 2019 Jul. 3; 10:833. doi: 10.3389/fphys.2019.00833. PMID: 31333491; PMCID: PMC6616128.
Zhang B, Zhu D W, Hu X J, Zhou M, Shang P, Lin S X. Human 3-alpha hydroxysteroid dehydrogenase type 3 (3α-HSD3): the V54L mutation restricting the steroid alternative binding and enhancing the 20α-HSD activity. J Steroid Biochem Mol Biol. 2014 May; 141:135-43. doi: 10.1016/j.jsbmb.2014.01.003. Epub 2014 Jan. 13. PMID: 24434280.

1) Method for the diagnosis of lipedema and/or for the individuation of treatments thereof with molecules capable of modulating the activity of AKR1C2 comprising at least one of the following steps:
(i) detecting step to identify rare and polymorphic variants in the sequence of AKR1C2 gene;
(ii) detecting step to quantify mRNA encoding an AKR1C2 isoform or to verify the presence of mRNA encoding an AKR1C2 polypeptide or fragment thereof;
(iii) detecting an increment or reduction of AKR1C2 enzymatic substrate or product or metabolites, in a biological sample of a lipedema patient compared to controls;
(iv) identifying natural and synthetic molecules capable of modulating AKR1C2 with possible therapeutic effect on lipedema.

2) The method for the diagnosis of lipedema and/or for the individuation of treatments thereof according to claim 1 wherein a criterion to predict disruptive missense variants in AKR1C1 and AKR1C2 is applied, the criterion comprising the evaluation of the following properties: the position's entropies, the predicted ΔΔG of the variant, and the positions' contribution to the overall substrate binding energy or to catalysis, wherein:
in the case of AKR1C2 the variants that concern residues with high contribution to the overall substrate binding energy, or that contribute to catalysis are favored and thus excluded, while in the case of AKR1C1 these variants are not favored and thus included;
then the entropy intended as conservation of a position and the predicted ΔΔG intended as the predicted change of the protein's fold stability when the variant is introduced are taken into account selecting a variant as potentially disruptive if it is located in a position with relative entropy >5 OR predicted ΔΔG<−3.

3) The method for the diagnosis of lipedema and/or for the individuation of treatments thereof according to claim 2 wherein during docking W227, L54, Y24 are set as flexible residues during docking.

4) The method for the diagnosis of lipedema and/or for the individuation of treatments thereof according to claim 1 wherein the variants of step (i) are selected from variants located upstream AKR1C2, in particular at the binding sites of retinoid acid-related orphan receptor and the glucocorticoid receptor and/or in regulatory regions of AKR1C1, AKR1C2, or AKR1C3 enzymes.

5) The method for the diagnosis of lipedema and/or for the individuation of treatments thereof according to claim 1 wherein the variants of step (i) are selected from the group consisting of rs145611933 (chr10:5019786); rs6601888 (chr10:4983447); rs36032941 (chr10:5020560); rs61856103 (chr10:5019349); rs4881378 (chr10:5022148); rs61856128 (chr10:5021190); rs10795227 (chr10:5025968); rs28571848 (chr10:5019979); rs34477787 (chr10:5071991); rs999611958 (His48Arg); Tyr55His; rs1462840208 (Trp86Ser); rs754792432 (His117Asp); rs778903438 (His117Pro); His117Arg; rs752532298 (Pro119Thr); and rs782186892 (Trp227Arg).

6) The method for the diagnosis of lipedema and/or for the individuation of treatments thereof according to claim 1 wherein the variants of step (i) are selected from rs143258520 that concerns a regulatory region upstream AKR1C1 or NP_995317.1:p.Ser320PhefsTer2 that concerns the C-term removal of AKR1C2.

7) The method for the diagnosis of lipedema and/or for the individuation of treatments thereof according to claim 1 wherein in step (ii) an AKR1C2 mRNA overexpression is detected.

8) The method for the diagnosis of lipedema and/or for the individuation of treatments thereof according to claim 1 wherein in step (iii) in a biological sample the DHT amount is determined and the determined amount identified as reduced compared to standard values.

9) The method for the diagnosis of lipedema and/or for the individuation of treatments thereof according to claim 1 wherein the selected variants and/or the wild-type variant of AKR1C2 and/or AKR1C1 are studied by functional modelling to verify their impact in terms of binding affinity to certain pharmaceutically active compounds.

10) The method for the diagnosis of lipedema and/or for the individuation of treatments thereof according to claim 1 wherein in step (iv) a natural or synthetic molecule is selected that inhibits the AKR1C2 activity without affecting the activity of AKR1C1 or less affecting the activity of AKR1C1, or in other words wherein in step (iv) a natural or synthetic molecule is selected that has a lower specificity for AKR1C1 than for AKR1C2.

11) A method of treating and/or preventing of human lipedema in a subject, the method comprising administering or applying to a subject in need thereof a therapeutically effective amount of a compound of natural or synthetic origin, preferably contained in a food supplement, cream or ointment, suitable for modulating the activity of AKR1C2.

12) The method according to claim 11 wherein the compound is an inhibitor of AKR1C2 or modulates the catalytic activity of the AKR1C2 enzyme, but is preferably no inhibitor of AKR1C1 and does not modulate the catalytic activity of its enzyme or at least has a lower specificity for AKR1C1 than for AKR1C2.

13) The method according to claim 12 wherein the compound comprises at least one of Triazolam, Sophoraflavanone B, Dydrogesterone, hPGS, Diazepam, Carbamazepine, Isoxanthohumol, Glycyrrhetinic Acid, Delorazepam, Paliperidone, Daidzein, Genistein, Cinacalcet, Bicalutamide, Ursodeoxycholic Acid, 6-Prenylnaringenin, Canaglifozin, Tauroursodeoxycholic Acid, Empaglifozin e Dapaglifozin.

14) The method according to claim 13 wherein the compound comprises Triazolam.

15) The method according to claim 11 wherein the compound is Triazolam, Alprazolam or Canrenone.

16) The method according to claim 11 wherein the step of administering or applying to a subject in need thereof a therapeutically effective amount of a compound of natural or synthetic origin is preceded by a step/steps for the diagnosis of lipedema that confirmed the tested person is affected by lipedema wherein said step/steps comprises at least one of the following steps:
(i) detecting step to identify rare and polymorphic variants in the sequence of AKR1C2 gene;
(ii) detecting step to quantify mRNA encoding an AKR1C2 isoform or to verify the presence of mRNA encoding an AKR1C2 polypeptide or fragment thereof;
(iii) detecting an increment or reduction of AKR1C2 enzymatic substrate or product or metabolites, in a biological sample of a lipedema patient compared to controls.


SEQUENCE LISTING (17 sequences)

SEQ ID NO: 1 - DNA, 19869 bp, Homo sapiens
  source 1..19869: mol_type = genomic DNA
aaacatttgccagccaggctagtgacagaaatggattcgaaatatcagtgtgtgaagctgaatgatggtcacttcatgcctgtcctgggatttggcacctatgcgcctgcagaggtaacaataatatttttagtgttgagagttcaaaggagctagagtaagtggaagctgaccaggttgtcaggcttgtgttcctatgttactctgcatgactcccctttaaacgtcagtctttgttctgcaatgccatcttgtcacagggtcatctactgcttatttgtggcactgttttgtcttctgtttatgtttatttcacagcttgtcagagtctatacaactcagcagaaagaacacggcttgcctgctccctctcttgaagattgattgcaatgggagtggtttctctgtttctttgtatagtcgaacagatatttacttcttccaagaagatacaattcagaggaatttttatggcaaaagttagtggagacggggtggatgaaatctgttgggagagtatcactgttctgatgggcggccttgagcaccacactgtaatactagacctggcctaaagagaatttctcatgtactattctcacctctgggaaaattacctgaaacgataaaatatcctcattttaagaaaaaaaaaatactagaacctaacaaggaaaatttaaattctttttctttgttaatatgggtcaaatttgtatcaaaatattgtaacagaaaaatttcttgtttctctgacaggaaaattaatggaatagattttcttttctagacttagctatttctacactggaagaaaacttgtatctcaagactgtcttgggaatacaatgtgtgtgaatagggactagttacataaaatttgggtttcacatttctgatctatacaatataaggatttacaaagggacattttgaagaccattgacaggagtggtttactttgtaatataaattgacatggagaggaagtaaagatgaggataccactaaagctccagctttggtaagcctttgagtactcatacagcaaagaaatgatcaaggatggtatgaggagagggctgtgagacccagtatctagttccttgtaattctatgggaaaaagaaagcctctgaaatactttccagataggaaaacagtttgctttgtttctgtttttgtcatttaaaagatatactttcaaatctgtatccaaatttgttacgtaagtcaataattgtaaatttcactgcaatcatatgtttcctaaaagagttgccttgaacttatggaactgaaccccattttgctaatcacttactccttgcagtctatttcttatacaaatcaatatccttagagtatacatttttacacctactatttagttctgtcatgccgacagaatggctaatggctgtgtagaaaagacttgacataatagactgctatccatagacaggcctgtttcccatataaacctgctggtatctgataaggtggatttctcactatttcctaactgataagaatggtggctgtaagtaactgcttgtgcaaactgcggagtttctgtgcacctgctttccttttggagacttttggaagttttgtgcctgcgaagcagagtgagtcaatgtaaccagtcccagacaaaatcttagtaatggagtatgtagagagcatctcttgtagacaacagttcacatacattgacaaaatatgattccgggtaaagtcagtgcattctgtgcaatatggtgggagaggaatcacaggagcttacactgtattccttcatatttccttccatgtggctatttcctcgtgttaatttctttttttgtttgtttggtttttgttttgtttttctgagatggagtctcgctttgtcgtccaggctggagtgcagtggtgcgacctcagctcactgcaagctccgcctcctgggttcacgtcattctcctgccccagcctccccagtagctgggactacaggcgcctgccaccatgcccggctaattttttctatttttagtagagatggagttgttaaccaggatggccttgatctcctgatctcatgatccgcccagctcggcctcccaaagtgctgggattacaggtgtgagccgccacgcctggccattccttgtgttaattttaccatactaaaaataattaagattatatctataactataggttgcatcctgttaatttttccaaaaaatgactgaacctgaaattgttttggggacacttccaaactagtggtcaagcagtgatttttctgggactgcagaagttcctgctgtgcccaacctttattactaactgggaaagacccagggagactgggatgggctcatgattctacatacagaactcatccaagaaaggaggaaaagctgatttttgtgaacgtcgctacttgtgcctgaactaactctcaggcacattagtcagaaaatactacctatggttactcccccaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaaggtactgtgcctatgatgagcttgtgtgcacatgtatttattgtgattgtgtggaggtggcagttctatgactggatccatagtatagggtgaattgtgcttatttattacgatttattcacacttactcatgtattaaaactaatatcaaaggcaggaagtgaagatggctttctcatctttgcagtgttccaattcatgccttcaaagtgcctttactttttgagctcagcacagatcaatatggtttaacatagactatagaatcagaaaaaatcctaatcatattatattatttcttacctcctgggtgagtttcttacacttgttatgattctcagtttccattattatgttgatcatagagagagaaagcaatataagcaagctctgtgagtattaaagaataacgcctacattatctcaaattgtgtccagcccaagttgacatattaaaaactctgtttgcttcctcagtttctaaaccacaaagatacttaatcttgcccattgggctgaatagatgaaataattacactactaacatgagttgtaaaacttaccaaagataattcagataatgggtatgcttatgatttgataacaacttttaccttttgagtaatttcactctttcattcaatatatgtactaactatatatccaacatattataaagttcatcatgatatggcagaacaaaaggcatcacaagaagagagagaataattttgcctgtggtcattagttttgaagtagtagaaaatgtctaaatattaggtggagcaaactagtaaaattggctcaagttttcattacacaacttcctttctctaacctctgcagctttggtgcaattcccatcgaccagagttggtccgaccagccttggaaaggtcactgaaaaatcttcaattggattatgttgacctctaccttattcattttccagtgtctgtaaaggtaggcagcttgtgtgatcaaattaatttcacttttgttctcagcataaatattgtttttatggatatttgaactaagcattttcttaggaggacatagggattataacatagaagaagaatcctaaatctaactcctaattcctttctatgggatacattttgaatccatacttccgtgattgcatgtctataagaaaagaaagtgcagaactctcaaagcctctgcctcaaaaacttgaggaaatgatagtcatctccttgaaggcacaaggtcttagttatgattcctgatttcacctcttgggatgttcacagacacagagtttcatgaagctgtggtgtccagaaaacctgctgcacatagggtgcacaatgagtttccatcttcttgcctctttttaaggggcgagaactcagtccgggagtgtcttaaactacagaccttcatgggaaaccttgttgcttctgcttcctctcttttcacactggaggttttatttttgcttagccatgaattcttgggtcattcataacttttgtcttaaggtactgaaaactagtcaggctagttaatgcaaaagggtatattagatatgataatgggaaatcaaagccagggctacattaagaatttgataaaaactctaaaaatatatcctttctccacccttatctctgctttattacaaaaggctttttaagtatttgttccaacctttttcgttggtggcatttatggctttggagcactgtcaggcccatgttcattaccgtgagctcctgtgcatctcctaatttccaaactagccttgaaaacacctccattgaccatgattggttcatggtcctgtgcatggaacatcatatgttcagggagataaagaactctgatagtggcacctgggtaaaaagtacaatccattatatctggatatcaagatcttttgcagttgaagagaggtattgccacagagaaaattataggagcagaagaaagtcaatgaaagtcaatgatgacactccattaggaaccagaaagaggtatttatttatacatgtaatagttgtaagagattagagggagcctgtcacctgaagacattccttataccttcatatgtagtacactctgtacataccactctctggagcactgcatcacctacctcatggaggattagtgtccttaaatgtacctcagagcatggctatgtgtggagaaattgacttgtgacatcactaaactgactgcttctacttcagccaggtgaggaagtgatcccaaaagatgaaaatggaaaaatactatttgacacagtggatctctgtgccacatgggaggtgagtgtttggaggtgagagaacggataagaaagatgaggtaggatacatctgtttcctatcttcttagtacaagtatggaaaatgcaccattggatcaaaaacttaggaactttacaaaggtagtttttgtgagcagtgaggaaaaagaaatgacaggcatacaaaagagagaagtggaggagaagaattgggggtaaggaggactgggatttatttccttgccttttcactaatctctccagtttcccaaggaggaaacagaaatacttactcttgcaatcactatcttcttccccaattttctgttttattttttcccttttaagaaccacggctagctagttttattgtcatatctagacagttgttactttcacagttctgtgtcacatttatcttgatcttccacacctcacaattcctttttcccaggccgtggagaagtgtaaagatgcaggattggccaagtccatcggggtgtccaacttcaaccgcaggcagctggagatgatcctcaacaagccagggctcaagtacaagcctgtctgcaaccaggtgagcaccctcagcctcctctcctttctgttcttcatgcccctctttctgtcctattgccaagtatccattcatttagtcccacttatctttgtaaaagagaagattctagagagcaaagactctgtctcgaagggcatagagggatcttacttgtacccggcttagaaaagtcttagaaaaggtgttgtaactttgatgctgaattgtgtgttatatttatgggaggtcaattcaatcaaggaggccaaggattgtcaattagactcagggaaaggtggacttacccctaagctatgaaagatgaccagagaatggatgggtgaaggtgatttggagggaactgtgcatagatatgaagccgtgaagagatggaatgaacagtacataaggagagagtgagagcaagagaagagaaatactagggattcacatgggcttcggatgtctcagtgcctcttggtgtctctgctttcacacttcttgaccaagggcatagatacactaaaaattatctgcagacaatgaaggtcatccatggtagaaagagaaaataacctgtttatgggagatatctgttcacagtggatgaaatccacactgaggaaggttcagcagaacatggagctgacctatgactgcccccgctcctagttggctgctcttctttggagtctgtcatgcaatcagctgcttaaacacatctattctctatgtgttccttttgtagcctagagataattccatcttttccttgagtcctgactggtgattccaggcctccttgatcaaattgactgtccccaaatgttacacacatttcagcattaaagttactacacttttcccagtaaattacatttttttgttttattttatgttttaaaacttagagtcaatcagtgaagatctacagtggaaatacatatgtataaaattagtgaatttggtataatgatacggtgctgtgataatttaataaaagcattgagaaattaatgtggttttaatactagtagttccccaattttttaatatggccattttcacactttccaaaaatttgataaaaataatactggagacccatgttaccatttatattcaaaattactaacattttgcaatgttatatggagtatatctgcataaatacatataatgtttctagaacctcttcaaggtaaattgtaaagatgacaattgacccctagagacctctcaaagcatgtcacaggaatgaagatattctcccagttccagtgtcattactgtatctaagaacatcatctatagtgccctcatgtcatttggtatccagtccatatttgaatttttccttgcttaatcaaaatatcttttgtagctcttctttttttaaccagaattcaattgtttttatattgttgtattttgatatcagttaacttgtttttcccagttgttgaaggtttctgtaaattagaagttagatgagagtgttttattacattgacttaaacctttggggcaagaaaatatttttatcatgctgtgtttttcaaattgcaccacactagactgcatacaatcacagaatgccccactagtggtgaaggtagttttaaaaacttggttaagctccaacaatgatagactggattaagaaaatgtggcacatatacaccatggaatactatgcagccataaaaaatgatgagttcatatcctttgtagggacatggatgaaactggaaatcatcattgtcagtaaactatcgcaagaacaaaaaaccaaacaccgcatattctcactcataggtgggaattgaacaatgagatcacatggatacaggaaggggaatatcacactctggggactgttgtggggtggggggaggggggagggatagcactgggagatatacctaatgctagatgacgagttagtaggtgcagcgtaccaacatggcacatgtatacatatgtaactaacctgcacaatgtgcacatgtaccctcgaacttaaagtataataaaaaaaaaaaaacttggttaagctaacagtggtcagatctgttgtttttaactcatgttttctctttaacaattactagtaatctgctgggggataattctcaacagaagaagtcatattcccatgacaaattttcacatatgattgagtatacatcagtgatttgaacttttttctactgcatttctgttttcttctttgttatttcttatttgttatttccttggggtttttacataatcatttacaattttaaccacttgtttaactattcttacctgacatttaattaaaaatgatttctcaccaagtaaaaataataaactactattcctctaagtgagggtaaaggtgaacttcttccttcaattagcaatgtactgtgaacgtaattactgtaatgaccaccatcagagtagacaaagattgttgttctttttctcattgtcgcactaattactacagactcacggattcttgtgttatgatttttaatttattttactcattttttaatgctcacatctactcagcatatatatattatatatatatatatataaaatatatatgaaagaagaatgtgatcaaagactcatgagggcaagtgtaaaagacaaaagcacttgtttcatagggaacccactggtgacatttgagatatcaacatacaaatccatcatacaaaaaggaaaagtaatgaaagactgtgaaaattatcagtatttatgtgtgtatgtatattttctagttcatctatatagatatatatcatagatggcatatatgatatatatattatatgtcagatcaattagaaaacatacatatacatgtatgcatttttggtattaattaggaaactagttcttttgtctttcagacctgccctcatgagtctttgaccaaattcttgatttctggcacgatttgatgcccaaggctcaaagaatcttacctcatcccagctctgaaattggccgtgtctcgttctagtggtcggagttattgagaaaccaggatagggttcatggagttgcttgtgctcagaaatagcatttctattttcttaggagagaggggcataaaatttatttttatgaaaaagatcatttttgacaataatattctcagttcaaatttaatgctctacattattcagcttcttttactttggtgattttaatattagtgtaacttttggattatctgatgcttttccatcttgctcgtctgcaggtggaatgtcatccttacttcaaccagagaaaactgctggatttctgcaagtcaaaagacattgttctggttgcctatagtgctctgggatcccaccgagaagaaccatggtaataagagatacaggaagtttacctaaaacaccggtttgataaaaaaaatttaatcagatcatgctgtttcctggagttcactcacagctgacttggggtgagggaagaatttgcatttctgacgagatcccaggtgatgttgaggctgctgttcgggggcctcgcttgagaagctccggtgcagagtggacgccttagtctgtttagggagccgcctaacaaactgtatccccagcctcagggcctcagcctttctgcctttccttccagggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgaggagcggggctgtgggcctcaggtctcctgcacagtgtccttcacacgtgtgcttcttgtaaggctctcaggacagccttgggccagctccatttccctgtatttcctatgcatgaactctttgtgtacgtcataagggtttcttctactctagcacaggagaggcaacagaggtggagagaaataggatgggatcaaaactacagatttgggttagcgttaagtcagtgatatccatatccctctgctggggccatgtccttttctttattttttataatgaagcattgattagatgttctttagtctccaactcatggcggatttttcttcttgtatgtttatgtagtataaattccttatatttattctcttagaattttatctttgcgaagtgcttgtttaactctgcctttttttttttggcgaggtggttgtgtgtctttatttttatgtgtaggagaccatatttataccaataccttaggatataagacaaacccacttaatatagaggcttaatgcaaaagcatttattgagctcaaggttccatggttagaaaattagcctgggctcagcctaaactcagtccattccttctgttcctggctgggctctcatgtgtgttcagggcagtgaggcagctctgcttcaggatgtccctggggcatctgtgtgagcgcccccctctcccatgcagttctttatcctccagccatccagcccagggttcacctcagcacagggcatgaccccaaggcagtgagtggacatgtgcaaagccttttcaggtctaggctcagagctgccaaccattgcttctgggaaattctatcacctaatgcaagttacaggacaggtcagactcaaggggtggggaaaaagacaccacctcttgttggaaggagctggaaattcatataaccaagcaagtggatacagggacatgtgattgttagagggcatgttcctaattaatctatcatttatacgatatgtgtgaaataacatattaatagttatatgtatgaatatgaatatatataaattgatatactatttcatatattcttaagaatacatccaagtctctaaagaattaatatccagaatacatgcatatatatgttttctaattagttatatacagatatatgaaatatcatatatgatatatattataaaacctatgtcacatatatgacatatatcatgtatatgtatatgtgtcactatttgctaaaacattaagtaaaatcagtgtagaaatccataggatactctgaaataatgatacagagaatgagaaagaaagtaaaaaatgacaatgaaacaattgatgaatctaggtaatggcatttactatattattttaaaaatatttttagacattagaaaacccacaaaactaaaaggaaatattatgaaataactgtggaaattatcagtatttatacatatgtgtatgttctctaattcatctagtatatatagatatatgacatatgtgttatataatatatatgttagattaactagaaaccatacatatatatgtatttattctggatattaattctttagagacttaaatgtattcttgaaataacatctcccaaattttacctgtgttgtcatattctctgtggttatgttttcacaaacaaaagtttctatccttcatgtagactaatttttcatgcctctttcttgtggttggtgagtgttgtgtcttatcctatattgggaatatcacaatatattactgtgttatcttacaaaattgttaatattctttccttataaatttggttaattctaatgggattgactttttaaatactgtatgagataaggatccaaatatgcatatctaatttttgaatttttagcaaaatttcccaccaacatttattgaaaagttcatatttgctcacataacttgatttttttacctgtaacatatatgaaatgtccatgagcaattatctggcctctcttccatcttcagttaacttatttacttttctgtactatgatgttaaaaagtaagtttcacttccagtatggtacaaatatttttctaacttcttcatttatttaataatatcttagctattcttgactaatgtgttatttaaaactttgaatcatcttattaacttccacaagcaaataaatgtttagtctcttgttgacattgtaataagcctccagatcaatgtggagagaactaaatgtttgacaatattgagatttccaatgcatgatcataatatgaatttcccacttattaatagccttttaataaatatataattaatttcataactttctctgttactgatgattttaatagtttttgataacttttcatttttgttttatggattttctaatatatagaaatattaaaagaataatgcaacaaatcttcttacctacattcatcaagtgttacattgctgtcattttttactctctttctctcattttctgtattactccttcagtttccctatggatttatacacttatttgattgaatgtattagaaagagtgacatctttttgatatttagagtctcccaaatttacatagtgagagtacccacaccttttgaatgtgaaatcgttagtatttagacaattattggcttgagtgatgttgaaaactaatttttcctattatacctgttcattggagagatagatattcaatgttattcatcaaacctactaaattatgctagacattacaaaatttgcctctagattcttgtgggtcttctaggtacatgaccctatcatgtgggcacaaagtaagctctgtttcttcttcagcttcctctgagatattctcttcatctgcagtgttgtgcagtttcaaaatattttatctgggaatggatttctgcttatttttgactggttttcattgaccacttgggtgtttagagaggtctttcataaattttggaaacgtatcaatcttttaagtattgtctctgcaccctactgtctgatgaacttggttgttcccaattggcaatgatgtaatctaaaaataattaaagttttctattactttgataggtgtttgaattccagttgacttcagaggagatgaaagccatagatggcctaaacagaaatgtgcgatatttgacccttgatatgtaagtaattttggagatggttgtcctaatttattttcagaggaggaacgtaggatgggtgttgagagtgacctccacaccaggggcacagaggccaacgtgagacagaggtgagacaggagctttctggaagtctcctcctggattcactccagagctctgttctctggcagggagagtggcctggggtcagcatgggtcaacctgtgcctctgctctcgtgactccaaggaactttccagagcagccaagatcattgctgaatctgcaccttccatgtaggcctgctgtttgtgctactgtctagtgtacacactgtggatattgcccatgtggtcgcattagatgtttccaaatctgtgcttctatcttgttcttcccaacctgctcaatgtcttaccagatcaaaggcagttccgtcaatcttgtgggccaggttccaattcccacctctttcacatggaattgcttgctggatcctgtcgatttagcatttttaatcatttcaaacctttttcgtgcctgtctttgcatgtttgcccatgagccaaacactgcatttgcctccagaaagtctgtcttagtgaagcgaataggagcacttggccttggtgttctgcaatatggagatctcagtgcagagaatgaagaaatgttaaaatcaaatgcagagtgtagatgcaaaagcaccattgttggagataatttataaccaataacagaagtttactattcctctgtgttctaaggttctaggattcatagaaggtcattcatataatgagggaaaaaagaagttatgtaactgctggcagtagggatttcaacaagtaatagagatgatgagataatgtgtgaaaaaaacaagcacaatggtacatcataagtacccaaaatttggtggatattattagtagtattttattcatttagaggaaaatacaaaatgaaaacggagatgataaaaagaaaaggaaaagtggcaagcattagaaagttttaaaaccaattaatgtttttggggatcatgaaatcaatcactagtgttattttgtagaactcaaatatggtctggagtaaaccacttgcgagcttccaagtgtttgctcttggtgtaatcaaaaaggataggctcaattccctaattaataagttgtaattgtacatgtgagcagtcatttatctgtgtctttacattttgtgttatttctgcctataaaatggtttatacattgtagctctttggatattggaccctatatcaaaaacaacaatttacatttctgaatctaaccaactatgttgcatatgataggcaggtagtaatatgtaaagtaattaagtataattgctaccagtataatcatcacactcaaaattgtttattagtgatgagcatatgtcttgcttattcctcttcaatgtaaggcagtatagttccatttttactgagaatttgaaatagaaaaggtcagtatatctctttgctctgttgacagatgtagagaaatatgtttgaacatttcactttgtgtgttttatgtgttaagttccaggcaggggaatagaattaaattattctttattttgaaaatacagatatgataaagtcacattcattaaacaaagaaacacagattctagagcagtcagaaaatgaacttcttaacatctacactagcggcagcttcctagaaatcactgcactacccgctagtaacggagtcattgccattcagagtgtgcatttttttttctctttccagttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctactttggtctccataacttctatgttttctttccttctgacacactagtgcccctaaattgtgatttgcctatacgtttagggccggggttggaagatgttaacaaccatttaagattcatttctgcagtgggagtgggtggagtttcaccctctgggaaaggggcaggtgacaggtatttatcagtcagtgcctctctagctcttgtaggaagaagcacacgcaggatggagtctagaggatgagcgatattgactagcaattcatgggctccctccagcagtgcgagggtcagagtttctggagccttgggaggaggcatccctgtgagggggggttagggagatgggagggcaccaggaaaagtgattagaagtcaggtatgggaaggctaaataggacagagtcgagtacatctctgcttggaaaaacatatcaacacccttttttttgaacattatatcttgctcataaaagaaaactttccacattgttttaacaaaccccacagctgagagtcaggcctgaatctttgatgtgtgcccattcacaacgttgaccctattggtttgtggtggggcagggcatcaaagacatcattgactaatcacattcccctgaatagctcatatttagaaaatattcttagattctaaaaatgtactattaatttgtgatattcagtcttttaaatattttatacattaaacaggcatagttacaaatataaaacaaaaatatcccaaagccattatgcatggcactcaagattaaaatgggaaataatacatctaataaatcaaatgttccaagacttcaaaggtcttttggaaacaggctatgtaaaacagcacactggtttcaaactttggtaaattttaagaacaactcttacaaaggcatttaattcttatacataattttcaggggacctaagttaatcagctaatcatgaagacatgattttcattttagaaaacacttttgaaaacttgggataatctcatgccttaatgatcaaagcattatgagaaggacagtggtttttaacctgggcatatgttctaacacatttactctccactattcgtactctggtagccatgttaaccccatcagagattccttctcaagccatgtctcagagctgagaggcatcccagcaagttttgcagctcacagttttttccgtaaattacttattctataaaattggagtaggccataaactttggagggccctagaccaattttttggattatttttcgtcttctatcattccgctgatcttagatattctctgcattaaatattaaatatcacttctaggctgaaaaatccccctaaaaatatttctagctcagatttttcctccaaattctgcaatagaagatcacaatgtgaactctgcatctccatgttaaagtctaatggacattcacacttagcatgtctcaaagaaatctcatgtaaaccatggccatcctgttctaccttaactttctgagtctatggaatgataatttcacatctcataaacttgactgatgtaagtgtcaagaaaagattgacattttgttaaaagttagtagtgaagtgtgtaacgcttaagcaaactttcatatttcaaatctctttagcaagtgtaactcttttttcaagatgtgaaataatcattaggtcagtcatttgtaaatagtacatctgctatggactttttccagttcttcaccatccatttttataaaactcttattgttaaaaaaaaagttactcagaatttcataaagccaaacacctgatttcaggaacacttgagatgtaagaaaattttatagggacctccaatcactaattttcctattttttctctcaaagaaatgctgaagggaggaattcaggttgaatgaaaggaaatagtaacttacagccatatagagttataaagacttcttgtaaatgtgaacatatggtaaaatataaaaacatgtatttttgaaattttggattctactcattattttacttcaatttaagatataaatgtatagaaataagtataattctaagctaatacgtatgcaatgtaggaagctgtaattactgaccaaaactatgtgaagtggagaaaacctggggaagtggatggttttagatgaaactgaagttaaattcatattgatttaaagtaaattgttataactttataaagtttttcatcatcaccacagcaatcacaaagagaataattatgaatatacgcaagaggaaatgagaagggaatccaaatgtcattaaaaaaaaatcacgccacctcacaaaatggtaacagtggatataaaggatagaaagctataaacctggtaggaaaacattgaaaatggcacaagtgactccttccctgtcaccaatgacagtcatgtgttgcataatgcctgggctatcttctgagaatggctttgttaggcaactgtgtcatggtggggacatgatagcattagagagaattctctcaaacactgtcatggccttatcaactatgcttctgtaatattctgaatccttcgctgcctttccaacagtgtttacaacggtttcctaagaaatgaaatccatttcaccaaaccacttttcttgctcatccataagaaacacttctcctttgttaaaattacatcatgagattgcaagcattcagtaacttgcaagctccaatttgaattctagttgtcttgctatttccaccacatctgcaattactttctccactgaaatcttgcattcctccaagtcatccaggagggttgaattcacttctttcaaactcctgttcaagtaaatgttttgccctcctgtcacgaatcatgaatgtttttaatggcatctagaatagtaaatctttttcagaagattttcaattttcttttcccaggttcttcagacaagtcaccatggtagctgtagacttatgaaatgtatttcctgaaccataaaacaagaacatcaaaattactctatgatccatgggaaacagaatgcatgttgtgttagcaggcatggaaagaacagtaatctctttcaacctctgtaagagagttcttggttgattaggtaccttgtcgataggcagttctaatttcaaagaactttttcttttgctgagcagtaagtcttaacagtgggcttaaaatattcagtaaaccattatttaaggagatgtggcttaaatatctaggctttcttgctttatttatagaacacttacagagtagatatagattaaatcttaaggaccctaggattgttagaatagtaaatgaacatcatatgttgtaatatagttagccagggaggggaaagatctctacaagaaataattttaaaaactactcaaagaaatcagagatatcacaaaatgacaaaacaagtacaggggcagcaaaaggtaacagatgtcaactatgagatctcatcaagaactaagaaaatggagaaaagtaccagatggcagatgggaggcagtgtgagcatgcctctcccacttggagattagtgtgtagagatttacattgtgaatttcttttcaagaaccaatgcagagacttaacagatatatgaaaaaatttcacatacactctgaagaagtctcaggcacaatcctactccatgaaataagaaaaaaccatcacatccccagattctgaggggagagagttcactccagagcatacatccccactggggatctggaaattcaggccacaagagaaggctttgatcctgtgcggacaagtgccttgagcagaatccacaggggagagctggaagtgcatggcagatatgagcacagaagctgggtgtccagccttgtgggcagaccgggagggatgtggcctgaaagccatggttattatctcagcgtgtaagctgatgatctgaggctggtcagagttctgtgcgcagactgcctggatgtaaacctggcactgttagcagagcactgcatgaatgaaaggaatgaaaatagccttgccaactgcataggtgctgggtgaggctcactgtggggggcgcacggcaagctattcaacattacgtacaggcatttgaggtcggggcatggaaaaatactgaggcagtgtgtgtatgttatttgtgctgagaatgaaactccttgaccttgaaaacaggacagggagtggtgtgtgtgctgtgataaggaatgctgaaaacggtctcctgagaatgcggtttgagtgcttttacaaggccacaggtgtctcacgacctgacctcataaagccatctagtggatgtttgtggcttaacaagccccttcaataagtacttggcggagggatgctggggggaccttcttagaagagctgcgccccacccccctccctggacagcccagctacaattgtctgagaactcactgcaagctgtaagcggcccactacttcccactcccttttttgaactcttctgtgcagcagaggcaattaaactctcctctggaacattaccccagcagcctgagaaccaccccttggcgctcacaggggctgcagcctgccttatccaaggacagttagagtgcagacccacctaaccctggacccactggtggtacccatccacccatcctggtagcttaacacaaagatacacttttaggagcttcatagcccctcttcccccattgcctgagaaaccactttccctgggcaacttagggcaagctcaaatcccactgctaccaccacagctggtgctcttttgcaagtgccacctcctagctggagaccaaccagcacagtccacacagcacctcaaggaagaataaatagaactgttcccaggaaggagaaaatgcctgcatgagctcagctgttaccactgcgtaccacaccctgaccagtcagaggtcttgagtcggtccgcatgacaagttcaccgctcgcataaccagcattcaagaaagccagcacactaagcctatctacagccagggagtctcagagtctacgtcactcccctgccacctcaatcagagctggtgctggtatccactgctgggagacttgtgctgaggtgaagcctgggctggatggctggaggatgaagaactgcacggaggagatggaggcacccagacagatgccccatggacatcctgaagcagagctgcctcactgccctgcacacacatgagagcgcagccaggaacagaaggactgggcaacattcaggcggagcccaggccaattttctaaacatggaatatgagttaaataaatggcttcgtattaaa

SEQ ID NO: 2 - DNA, 6513 bp, Homo sapiens
  source 1..6513: mol_type = genomic DNA
atggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacatgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctactttggtctccataacttctatgttttctttccttctgacacactagtgcccctaaattgtgatttgcctatacgtttagggccggggttggaagatgttaacaaccatttaagattcatttctgcagtgggagtgggtggagtttcaccctctgggaaaggggcaggtgacaggtatttatcagtcagtgcctctctagctcttgtaggaagaagcacacgcaggatggagtctagaggatgagcgatattgactagcaattcatgggctccctccagcagtgcgagggtcagagtttctggagccttgggaggaggcatccctgtgagggggggttagggagatgggagggcaccaggaaaagtgattagaagtcaggtatgggaaggctaaataggacagagtcgagtacatctctgcttggaaaaacatatcaacacccttttttttgaacattatatcttgctcataaaagaaaactttccacattgttttaacaaaccccacagctgagagtcaggcctgaatctttgatgtgtgcccattcacaacgttgaccctattggtttgtggtggggcagggcatcaaagacatcattgactaatcacattcccctgaatagctcatatttagaaaatattcttagattctaaaaatgtactattaatttgtgatattcagtcttttaaatattttatacattaaacaggcatagttacaaatataaaacaaaaatatcccaaagccattatgcatggcactcaagattaaaatgggaaataatacatctaataaatcaaatgttccaagacttcaaaggtcttttggaaacaggctatgtaaaacagcacactggtttcaaactttggtaaattttaagaacaactcttacaaaggcatttaattcttatacataattttcaggggacctaagttaatcagctaatcatgaagacatgattttcattttagaaaacacttttgaaaacttgggataatctcatgccttaatgatcaaagcattatgagaaggacagtggtttttaacctgggcatatgttctaacacatttactctccactattcgtactctggtagccatgttaaccccatcagagattccttctcaagccatgtctcagagctgagaggcatcccagcaagttttgcagctcacagttttttccgtaaattacttattctataaaattggagtaggccataaactttggagggccctagaccaattttttggattatttttcgtcttctatcattccgctgatcttagatattctctgcattaaatattaaatatcacttctaggctgaaaaatccccctaaaaatatttctagctcagatttttcctccaaattctgcaatagaagatcacaatgtgaactctgcatctccatgttaaagtctaatggacattcacacttagcatgtctcaaagaaatctcatgtaaaccatggccatcctgttctaccttaactttctgagtctatggaatgataatttcacatctcataaacttgactgatgtaagtgtcaagaaaagattgacattttgttaaaagttagtagtgaagtgtgtaacgcttaagcaaactttcatatttcaaatctctttagcaagtgtaactcttttttcaagatgtgaaataatcattaggtcagtcatttgtaaatagtacatctgctatggactttttccagttcttcaccatccatttttataaaactcttattgttaaaaaaaaagttactcagaatttcataaagccaaacacctgatttcaggaacacttgagatgtaagaaaattttatagggacctccaatcactaattttcctattttttctctcaaagaaatgctgaagggaggaattcaggttgaatgaaaggaaatagtaacttacagccatatagagttataaagacttcttgtaaatgtgaacatatggtaaaatataaaaacatgtatttttgaaattttggattctactcattattttacttcaatttaagatataaatgtatagaaataagtataattctaagctaatacgtatgcaatgtaggaagctgtaattactgaccaaaactatgtgaagtggagaaaacctggggaagtggatggttttagatgaaactgaagttaaattcatattgatttaaagtaaattgttataactttataaagtttttcatcatcaccacagcaatcacaaagagaataattatgaatatacgcaagaggaaatgagaagggaatccaaatgtcattaaaaaaaaatcacgccacctcacaaaatggtaacagtggatataaaggatagaaagctataaacctggtaggaaaacattgaaaatggcacaagtgactccttccctgtcaccaatgacagtcatgtgttgcataatgcctgggctatcttctgagaatggctttgttaggcaactgtgtcatggtggggacatgatagcattagagagaattctctcaaacactgtcatggccttatcaactatgcttctgtaatattctgaatccttcgctgcctttccaacagtgtttacaacggtttcctaagaaatgaaatccatttcaccaaaccacttttcttgctcatccataagaaacacttctcctttgttaaaattacatcatgagattgcaagcattcagtaacttgcaagctccaatttgaattctagttgtcttgctatttccaccacatctgcaattactttctccactgaaatcttgcattcctccaagtcatccaggagggttgaattcacttctttcaaactcctgttcaagtaaatgttttgccctcctgtcacgaatcatgaatgtttttaatggcatctagaatagtaaatctttttcagaagattttcaattttcttttcccaggttcttcagacaagtcaccatggtagctgtagacttatgaaatgtatttcctgaaccataaaacaagaacatcaaaattactctatgatccatgggaaacagaatgcatgttgtgttagcaggcatggaaagaacagtaatctctttcaacctctgtaagagagttcttggttgattaggtaccttgtcgataggcagttctaatttcaaagaactttttcttttgctgagcagtaagtcttaacagtgggcttaaaatattcagtaaaccattatttaaggagatgtggcttaaatatctaggctttcttgctttatttatagaacacttacagagtagatatagattaaatcttaaggaccctaggattgttagaatagtaaatgaacatcatatgttgtaatatagttagccagggaggggaaagatctctacaagaaataattttaaaaactactcaaagaaatcagagatatcacaaaatgacaaaacaagtacaggggcagcaaaaggtaacagatgtcaactatgagatctcatcaagaactaagaaaatggagaaaagtaccagatggcagatgggaggcagtgtgagcatgcctctcccacttggagattagtgtgtagagatttacattgtgaatttcttttcaagaaccaatgcagagacttaacagatatatgaaaaaatttcacatacactctgaagaagtctcaggcacaatcctactccatgaaataagaaaaaaccatcacatccccagattctgaggggagagagttcactccagagcatacatccccactggggatctggaaattcaggccacaagagaaggctttgatcctgtgcggacaagtgccttgagcagaatccacaggggagagctggaagtgcatggcagatatgagcacagaagctgggtgtccagccttgtgggcagaccgggagggatgtggcctgaaagccatggttattatctcagcgtgtaagctgatgatctgaggctggtcagagttctgtgcgcagactgcctggatgtaaacctggcactgttagcagagcactgcatgaatgaaaggaatgaaaatagccttgccaactgcataggtgctgggtgaggctcactgtggggggcgcacggcaagctattcaacattacgtacaggcatttgaggtcggggcatggaaaaatactgaggcagtgtgtgtatgttatttgtgctgagaatgaaactccttgaccttgaaaacaggacagggagtggtgtgtgtgctgtgataaggaatgctgaaaacggtctcctgagaatgcggtttgagtgcttttacaaggccacaggtgtctcacgacctgacctcataaagccatctagtggatgtttgtggcttaacaagccccttcaataagtacttggcggagggatgctggggggaccttcttagaagagctgcgccccacccccctccctggacagcccagctacaattgtctgagaactcactgcaagctgtaagcggcccactacttcccactcccttttttgaactcttctgtgcagcagaggcaattaaactctcctctggaacattaccccagcagcctgagaaccaccccttggcgctcacaggggctgcagcctgccttatccaaggacagttagagtgcagacccacctaaccctggacccactggtggtacccatccacccatcctggtagcttaacacaaagatacacttttaggagcttcatagcccctcttcccccattgcctgagaaaccactttccctgggcaacttagggcaagctcaaatcccactgctaccaccacagctggtgctcttttgcaagtgccacctcctagctggagaccaaccagcacagtccacacagcacctcaaggaagaataaatagaactgttcccaggaaggagaaaatgcctgcatgagctcagctgttaccactgcgtaccacaccctgaccagtcagaggtcttgagtcggtccgcatgacaagttcaccgctcgcataaccagcattcaagaaagccagcacactaagcctatctacagccagggagtctcagagtctacgtcactcccctgccacctcaatcagagctggtgctggtatccactgctgggagacttgtgctgaggtgaagcctgggctggatggctggaggatgaagaactgcacggaggagatggaggcacccagacagatgccccatggacatcctgaagcagagctgcctcactgccctgcacacacatgagagcgcagccaggaacagaaggactgggcaacattcaggcggagcccaggccaattttctaaacatggaatatgagttaaataaatggcttcgtattaaa

SEQ ID NO: 3 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 4 - protein, 297 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 5 - DNA, 19869 bp, Homo sapiens; identical to SEQ ID NO: 1
  source 1..19869: mol_type = genomic DNA

SEQ ID NO: 6 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDLFAGPPNYPFSDEY

SEQ ID NO: 7 - DNA, 16083 bp, Homo sapiens
  source 1..16083: mol_type = genomic DNA
gctaaccaggccagtgacagaaatggattcgaaataccagtgtgtgaagctgaatgatggtcacttcatgcctgtcctgggatttggcacctatgcgcctgcagaggtaacaataatatttttagtgttgagagttcaaaggagctagagtaagtggaagctgaccaggttgtcaggcttgtgttcctatgttactctgcatgactcccctttaaacgtcagtctttgttctgcaatgccgtcttgtcacagggtcatctactgcttatttgtggcactgttttgtcttctgtttatgtttatttcacagcttgtcagagtctataaaactcagcagaaagaacacggcttgcctgctccctctcttgaagattgattgcaatgggagtggtttctctgtttctttgtatagtcgaacagatatttacttcttccaagaagatacaattcagaggaatttttatggcaaaagttagtggagacggggtggatgaaatctgttgggagagtatcactgttctgatgggcggccttgagcaccacactgtaatactagacctggcctaaagagaatttcccatgtactattctcacctctgggaaaattacctgaaataataaaatatcctcattttaagaaaaaaaaaaaaaaactagaaccaaacaaggaaagtttaaattctttgtctttgttaatatgggtcagatttgtatcaaaatattgtaacagaaaaatttcttgtttctctgacaagaaaattaatggaatatattttattttctagatttagaaatagctaaatgctatttctacactggaagaaaacttgtatctcaagactgtcttgggaatacaatgtgtgtgaataggaactagttatataaaatttgagtctcacatttctcatctataagacataaggagttacaaagggacattttgaagaccattgacaggagtggtttattttgtaatataaattggcatggagaggaagtaaagatgaggataccactgaagctccggccttggtaagcctttgattgcttgcacagcgaagagaagatcaaagatggtatgaggagagggctgtgagacccggtatctagttccttgtaatttcacaggaaaaagcctctgaaatactttcaaggtgggaagatagtttgctttgtttctgtttttgtcatttaaaagatatactgtcaaatctgtatccaaatttgttacataagtcaataattgtaaattttactgcaatcatatgtttcctgacagagttgccttgaatttatggagctgaaccccattttgctaatcacttactccctgcagcctatttcttacatacacaaatcaatatccttagagtatacatctttactcctagtatttggttctgttatgcagacagaatggccaaaacctgggtagaaaagatctaacataatagactgctatccatagatgtgcctatttccaatataaacctgctggcatctgagaaagtggagttctccctgtttcctaactgataagaatggtggctctacgtaactgtttgtgtaaactgtggggtttctgtgcacctgctttgcttttggagactggaagatttgtgtctgctaggcagagtgagtcaatgtatccagtcccagacaaaatcttagcaatggagtatgtagagggcatctcttgtagacaacagttcacatacattgacaaaatatgatgctggataaagtcagtgcgctctgtgcaacatggtgggagaggaatcacaggagcttacactgcattccttcataattccttccatgtggctatttccttgtgttaatttcaccatactaaaaataattaagattatgtctgtaactatagttgcgtcctgttgatttttccaaaaaatcactgaaagtgaaattgttttggggacacttccaagctagtggtcaagcagtgatttttctgggactgcagaagttcctgctatgcccaacctttattactaaatgggaaagacccagtcagactgggatgggcttatgattctacatacagagctcatccaagaaaggaggaaaagctgatttttgtgaacgttgctgcttgtgcccgaactaactctcaggcacattagtcagaaaatattacgtttggttgctcccccaggttcctaaaagtaaagctctagaggccgtcaaattggcaatagaagccgggttccaccatattgattctgcacatgtttacaataatgaggagcaggttggactggccatccgaagcaagattgcagatggcagtgtgaagagagaagacatattctacacttcaaaggtactgtgcctatgatgagcttgtgtgcacatgtatttattgtgattgtgtggaggtggcagttctatgactggatccatagttgagggtgaattgtgcttatttatttcgatttattcacacttactcatgtattaaaactaatatcaaaggcaggaagtgaagatggctttctcatctttgcagtgttccaattcatgccttcaaagtgcctttactcttcgagctcagcacagatcaatatggtttaacatagactatagaatgagagataatcttaatcatattatgttatctgttacctcctgggtgagtttcttacatttgttatgattctgagtttccattgtcattttgatcagagagagagagaaagcgatataagagagctcagtgagtataaaagagtgatacctacattgttttaaattgcatccagcccaagttaacatattaaaaactgtgtttgcctcctcagtttccaaaccacaaagatacttagtcttgcccattgggctgaatagatgaaataattacaccactaatatgagttgtaaaacttaccaaagataattcagataatgggtatgcttatgatttgataacaacttttaccttttgagtaatttcactctttcattcaatatatgtactaactatatatccaacatattataaagttcatcatgatatggcagaacaaaaggcatcacaagaagagagagaataattttgcctgtggtcattagttttgaagtagtagaaaatgtctaaatattaggtggagcaaactagtaaaactggctcaagttttcattacacaacttcgtttctctaacctctgcagctttggagcaattcccatcgaccagagttggtccgaccagccttggaaaggtcactgaaaaatcttcaattggactatgttgacctctatcttattcattttccagtgtctgtaaaggtaggcagcttgtgtgatcaaattaatttcacttttgttctcagcataaatattgttttcatggagatttgaactaagctttttcttaggaggacatagggattttaacatggaagaagagccctaaacataactcctaattcctttctatggaacagaaagcaattttgaatccatacttccgtgattgcatgtctacaagaaaagagagtgcagaatcctcaaagcctctgcctcaaaaacttgaggaaatgacaatcatctccttgaaggcacaaggtcttatttatgattcctgatttcacctcttgggatgttcacagacacagagtttcatgaagctgtggtgtccagaaaacctgctgcacatagggtgcacaatgagtttccatcttcttgcctcttttcaaggggcaagaactcagtccgggaatgtcttaaactacaaaccttcatgggaaaccttgttgcttctgcttcctctcttttcacactggaggttttatttttgcttagccatgaattcttgtgtcattcataacttttgtcttaaggtactgaaaactagtcaggctagttaatgcaaaagggtatattagatatgataatgggaaatcaaagccagggctacattaagaatttgataaaaactctaaaaatatatcctttctccacccttatctctgctttattacaaaaggctttttaaatatttgttccaacctttttcgttggtggcatttatggctttggagcactgtcaggcccatgttcattaccgtgagctcctgtgcatctcctaatttccaaactagcctggaaaacgcctccattgaccatgattggttcatggtcctgtgcatggaacatcatatgttcagggagataaagaactctgatagtggcacctgggtaaaaagtacaatccattatatctggatatcaagatcttttgcagttgaagagaggtattgccacagagaaaattataggagcagaagaaagtcaatgaaagtcaatgatgacactccattaggaaccagaaagatggtatttatttatacatataataggtgtaagagattagaggaagcctgtcacctgaagacattccttataccttcatatgtagtacactttgtacataccactctctggagcgctgcatcacctacctcatggaggattagtgtccttaaatgtacctcagagcatggctatgtgtggagaaattgacttgtgacatcactaaactgactgcttctacttcagccaggtgaggaagtgatcccaaaagatgaaaatggaaaaatactatttgacacagtggatctctgtgccacatgggaggtgagtgtttggaggtgagagaacggataagaaagatgaggtaggatacatctgtttcctatcttcttagtacaagtatggaaaatgcaccattggatcaaaaacttaggaactttacaaaggtagtttttgtgagcagcgaggaaaaagaaatgacaggcatacaaaagagagaagtggaggagaagaattgggggtaaggaggactgggatttctttccttgcctgtacattaatctctccactttcctaaggaggaaacagaaattctcactcttgcaatcactatcttcttccccaatttttctgttttattttctcccttttaagaaccacggctagctagtttgattgtcacgtctagacagttgttgctttcacagttctatgttacatttatcttgctcttcagtcgactacacaaatgatacctcacaattcctttttcccaggccatggagaagtgtaaagatgcaggattggccaagtccatcggggtgtccaacttcaaccacaggctgctggagatgatcctcaacaagccagggctcaagtacaagcctgtctgcaaccaggtgagcgccctcagcctcctctcctttctgttcttcatgcccctctttctgtcctattgccaagtatccattcatttagtcccacttatctttgtaaaagagaagattctagagaacaaagcctcttcctagaagggcatggagtcctcttacttgtaccctgcttagaaaagtcttacgaaaaggtgttgtaactttgatgctgaattgtgtgttacatttataggaggtcaattcgatcaagaagtccaaggattgccaattagactcagggaaaggtggacttacccctaagctacgaaagatgaccagagagtggacgggtgaaggtgatttggagggaactgtgcatagatatgaagccatgaagagatggaatgaacagtacataaggagagagtgagagcaagagaagagaaatactagggattcacatgggcttcggatgtctcagtgtctcttgatatctctgctttcacacttcttgaccaagggcatagatacactaaaaattatctgcagacagtgaaggtcatccatggtagaaagagaaaatgacctgtttatggagatatctgttcacagtggatgaaatccacactgaggaaggttcagcagaacatggagctgacctatgactgccccgctcctagtcggctgctttttggagtctgtcatgcaatcagctgcttaaacacatctattctatgcatggttctttcatagcctagagataattccatcttttccttgagtcctgactggtgattccaggcctccttgatcaaattgactgcccccaaatgttacacacatttcagcattaaaatcactacacctttcccagtaacttacattttttgttataattttgttataatttggtataatgatatggtgctgtgataatttaataaaagcattgagaaattaatgtggttttaatactagtagttccccaattttttaatatggccattttcacactttccaaaaatttgataaaaataatactggagacccatgttaccatttatattcaaaattactaacattttgcaatgttatatggagtatatctgcataaatacatataatgtttctagaacctcttcaaggtaaattgtaaagatgacaattgacccctagagacctctcaaagcatgtcacaggaatgaagatattctcccagttccagtgtcattactgtatctaagaacatcatttatagtgcccttatgtcatttggtatccggtccatattttaatttttcattgcttaatcaaaatatcttttgtagctcttctttttttaaccagaattcaattgtttttatattgttgtatttgatatcagttaacttgtttttcccagttgaaggttcctgtaaattaaaagttagatgtgagtattttattacattggcttaaaactttggggcaagaaaatgtttttatcatgctgtgtttttcaaattgcacgacactagactgcatacaatcacagaatgccccactagtggtgaaggtagttttaaaaacttggttaagctaacagtggtcagatctgttgtttttaactcatgttttctctttaacaattactagtaatctgctgggggataattctcaacagaagaagtcatattcccatgacaaattttcacatatgattgagtatacatcagtgatttgaacttttttctactgcatttctgttttcttctttgttatttcttatttgttatttccttggggtttttacataatcatttacaattttaaccacttgtttaactattcttacctgacatttaattaaaaataatttctcaccaagtaaaaataataaactactattcctctaagtgagagtaaaggtaaacttcttccttcaattagcaatgtactgtgaacgtaattactgtaatgaccaccatcagagtagacaaagattgttgttctttttctcattgtcgcactaattgctacagactcacggattcttgtgttatgatttttaatttattttactcattttttaatgctcacatctactcagcatatatatatatattatatatatataatatatatgaaagaagaatgtgatcaaagactcatgagggcaagtgtaaaagacaaaagcacttgtttcatagggaacccactggtgacatttgagatatcaacatacaaatccatcatacaaaaaggaaaagtaatgaaagactgtgaaaattatcagtatttatgtgtatatgtatattttctagttcatctatatagatatatatcatagatggcatatatgatatatatatgtcagatcaattagaaaacatacatatatatgtatgcatttttggtattaattaggaaactagttcttttgtttttcagacctgccctcatgagtctttgaccaaattcttgatttctggcacgatttgatgcccaaggctcaaagaatcttacctcatcccagctctgaaattggccgtgtctccttctagtggtcggagttattgagaaaccaggatagggttcatggagttgcttgtgctcagaaatagcatttctattttcttaggagagaggggcataaaatttatttttatgaaaaagatcatttttgacaataatattctcagttcaaatttaatgctctacattattcagcttcttttactttggtgattttaatattaatgtaacttttggattatctgatgcttttccatcttgctcgtctgcaggtggaatgtcatccttacttcaaccagagaaaactgctggatttctgcaagtcaaaagacattgttctggttgcctatagtgctctgggatcccatcgagaagaaccatggtaataagagatacaggaagtttacctaaaacactggtttgataaaaaaaatttaatcagatcatgctgtttcctggagttcactcacagctgacttggggtgagggaagaatttgcatttctgacgagatcccaggtgatgttgaggctgctgttcgggggcctcgcttgagaagctccggtgcagagtggacgccttagtctgtttagggagccgcctaacaaactatcgccagcctcagggcctcagcctttctgcctttccttccagggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgaggagcggggctgtgggcctcaggtctcctgcacagtgtccttcacacgtgtgcttcttgtaaggctctcaggacagccttgggccagctccatttccctgtatttcctatgcatgaactctttgtgtacgtcataagggtttcttctactctagcacaggagaggcaacagaggtggagagtaataggatgggatcaaaactacagatttgggttagcgttaagtcagtgatatccatatccctctactggggccatgtccttttctttattttttataatgaagcattgattagatgttctttagtctccaactcatggcggatttttcttcttgtatgtttatgtagtataaattccttatatttattctcttagaattttatctttgcgaagtgcttgtttaactctgcctttttttttggcgaggtggttgtgtgtctttatttttatgtgtaggagaccatatttataccaataccttaggatataagacaaacccacttaatatagaggcttaatgcaaaagcatttattgagctcaaggttccatggttagaaaattagcctgggctcagcctaaactcagtccattccttctgttcctggctgggctctcatgtgtgttcagggcagtgaggcagctctgcttcaggatgtccctggggcatctgtgtgagtgcccccctctcccatgcagttctttatcctccagccatccagcccagggttcacctcagcacagggcatgaccccagcatagtgaatggacatgtgcaaagcctttttaggtctaggctcagagacaccaaccattgcttctgggaaattctatcacctaatgcaagttacaggacaggtcagactcaaggggtggggaaacacacaccacctcttactggaaggaactggaaattcatatagccaagcaagtggacacagggacatgtgattgttagagggcatgttcctaattaatctatcatttatatgatatgtgtgaaataatatatcaatagttatatgtatgaatatgaatatatatagatatattatttcatatattctcaagaattcatacaagtctctaaagaattaacatccagaatacatacatatgtgtatgttttctaattagttagatacataaaatatacatcacgtatgatatatattataaaacctatgtcatatatatgacatatatcatgtatatgtatatacgtcactatttactaaaacattaagtaaaataagtgaagaaatccataggatactctgaaatagtgatacagagaacgagagaataaaaaatgacaataatgaaacaattgatgaatctaggtaatggcattcattaaattattttaaaaatatttttggacattagagaattcacaaaacaaaaatgaaaagttatgaaagactgtggaaattatcagtatttatacatatgtgtatgttctctaattcatctagtatatatagatatatatgatatatgacatatgtgttatataatatatatgttagattaactagaaaccatacatatatatgtatttattctggatatgaattctttagagacttgaatgtattcttgaaataacatctcccaaattttatctgcgttgtcatattctctgtggttatgttttcacaaacaaaagtttctatccttcatgtagactaatttttcatgcctctttcttgtggttggtgtgtgttgtgtcttatcctatattgggaatatcacaatattttactgtgttatcttacaaaattgttaatattctttccttataaatttggttaattctaatgggtatgacttttaaaatattatgtgaaataaggatccaaatatgcgtatctaatttttgaatttttagcaaaatttaccatttattgaaaagtttatatttgctcacataacttgaatttttttacctgacacatatatgaaatgtccatgagcaattatctggcctctcttccatcttcagttaacttattcacttttctgtgctataatgttaaaaagtaagtttcacttccagtatggtacaaatatttttctaacttcttcatttatttaataatatcttagctattcttgactaatgtgttatttaaaattttgaatcatcttattaacttccacaggcaagtaaatgttttagtctctcattgacattgtaataagcctgcagatcaatgcagagagaactgaatctttgacaatattgagatttccagtccatgatcataatatgaatatcccactacttattaataaatcatctcttaataaatatatcattaattgcataaatttctccattactgatcattttttttttgagacagagtctcgttctgtcacccagggtggagtgcagtggcacgaactcggctcactgcaacctccacctcccgggttcaagcagttctcctgcctcagcctccagagtagctgggactgctggcgcatgctgccatgcccagctaattttttgtattttagtagagacgggtttcactgtgttgcccaggctgttctctaactcctgaaatcaggcaatcctcctgcgctggcctcccaaagtgctaggattataggcgtgagccaacgtgcccggcaattactgataattttaatagtttttgataacttttcattttcattttgtggattttctaatatatagaaatattaaaagaataatgcaacaaatcttcttacctacattcatcaagtgttacattgctgtcattttttactctctttctctcattttctgtattactccttcagtttccctatggatttatacacttatttgattgaatgtattagaaagagtgacatctttttgatatttagagtctcccaaatttacatagtgagtacccacaccttttgaatgtgaaatcgttagtatttagacaattattggcttgagtgatgttgaaaactgatttttcctattatacctgttcattggagagatagatattcaatgttattcatcaaacctactaaattatgctagacattacaaaatttgcctctagattcttgtgggtcttctaggtacatggccctatcatgtgggcacaaagtaagctctgtttcttcttcagcttcctctgagatattctcttcatctgcagtgttgtgcagtttcaaaatattttatctgggaatggatttctgcttatttttgactggttttcattgaccacttgggtgtttagagaggtctttcataaattttggaaacgtatcaatcttttaagtattgtctctgcaccctactgtctgatgaacttggttgttcccaattggcaatgatgtaatctaaaaataattaaagttttctattactttgataggtgtttgaattccagttgacttcagaggagatgaaagccatagatggcctaaacagaaatgtgcgatatttgacccttgatatgtaagtaattttggagatggttgtcctaatttattttcagaggaggaacgtaggatgggtgttgagagtgacctccacaccaggggcacagaggccaacgtgagacagaggtgagacaggagctttctggaagtctcctcctggattcactccagagctctgttctctggcagggagagtggcctggggtcagcatgggtcaacctgtgcctctgctctcgtgactccaaggaactttccagagcagccaagatcattgctgaatctgcaccttccatgtaggcctgccgtttgtattactgtctagtgtacacactgtggatatggcccatgtggtcgcattagatgtttccaaatctgtgcttctatcttgttcttcccaacctgctcaatgtcttaccagatcaaaggcagttccgtcaatcttgtgggccaggttccaattcccacctctttcacatggaattgcttgctggatcctgtcaatttagcatttttaatcatttcaaacctttttcgtgcctttctttgcatgtttgcccatgagccaaatactgcatttgcctccagaaagtctgtcttagtggagcgaataggagcacttggccttggtgttctgcaatatggagatctcagtgtagagaatgaggaagtgttaaaatcaaatgcagagtgtagatgcaaaagcaccattattggagataatttataaccagtaacagacgtttactattcctctatattctaaggttctaggattcatagaaggtcattcaaataatgagggaaaaagaagttatgtaactgatggcagtggagattttaacaagtaatagagatgatgagataatgtgtgaaaaaaacaagcacaatggtacatcataagtacccaaaatttggtggatattattagtagtattttatccacttagcagaaaatacaaaatgaaaatggaaatgataataagaaaaggaaaagtggcaagcattagaaatgtttacaactgattaatgttttttatgatcatgaaatcaatcactagtgttattttgtagaactcaaatatggtctggagtaaaccacttgcgagcttccaagtgtttgctctcggtgtaatcacaaaggataggctcaattccgtaattaacaagttataactgtatatgtgagcagtcatttatctgtgtctttacattttggattatgtctacctataaaatggtttatgcattgtagctctttgaatattgggccctataacataaacaataatttacgtttctaatctaaccaagtgtgttgcgtatgataggcaggtagtaagtaatatgtaaagtaataaaataaagtacaattactaccagtataattatcacactcaaaattgtttattagtgatgagcatatatcttccttattcctcttcaatgtaaggcagaaggagttcctgtttttagtgagaatttgaaatagaaaaggtcagaatatctctttgttgacagatgtgggatctgttgttgagagatgtagagaaacatgtttgaacatttcactttgtgtgttttatgtgttaagttccaggcaggggaatagaattaaattattctttattttgaaaatacagatatgataaagtcacgttcattaaacaaagaaacacagattctagagcagtcagaaaatgaacttcttaacatctacactagcggcagcttcctagaaatcactgcgctacgggctagtaacggagtcattgccattcagagtgtgcatttttttttctctttccagttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggcgacttcagtaagctacagctaagcccatcggccggaaaagaaagacaataattttgtttttcattttgaaaaaattaaatgctctctcctaaagattcttcacctactttggtctccataacttctatgttttctctccttctgacacactagtgcccccaaattgtgatttgcctatacgtttagggccggggttggaagatgttaacaaccatttaagattcatttctgcagtgggagtgggtggagtttcaccctctgggaaaggggcaggtgacaggtatttatcagtcagtgcctctctagctcttgtaggaagaagcacacgcaggatggagtctagaggatgagcgatattgactagcaattcatgggctccctccagcagtgcgagggtcagagtttctggagccttgggaggaggcatccctgtgagggggggttagggagatgggagggcaccaggaaaagtgattagaagtcaggtatgggaaggctaaataggacagagtcgagtacatctctgcttggaaaaacatatcaacacccttttttttgatcattatatcttgttcataaaagaaaactttccacattgttttaacaaaccccacagctgagagtcaggcctgaatctttgatgtgtgcccattcacaacgttgaccctattggtttgtggtggggcaggacatcgaagatatcattgactcatcacattcccctgaatagctcatatttagaaaatattcttagattgtaaaaatgtactgttcatttgttatatgcaatcttttaaatgttttatactttaaacaaggcatagttacaagtataaaacataaatatcccaaagccattatgcatggcactcaagattaaaatgggaaataatacatctaataaatcaaatgttccaagacttcaaatgtcttttggaaacaggctatgtaaaacagcacactggtttcaaactttggtaaattttaagaagaactcttacaaaggcatttaattcttatacataattttcaggggaactaatcaaatcagctaatcatgaagacatgattttcgttttagaaaacacttttgaaaacttgggataatctcatgtcttaatgatcaaagcattatgagaaggacagtggttttttacctgggcacactttctaacacatttactctccactattcgtactctggtagccatgttaaccccatcagagattccttctcaagccatgtctcagagctgataggcatcccagcaagttttgcagctcacaatttttctgtaaattacttattctataaaattggaagaggccataaactttggagggccctagaccaattttttggattatttctggtctactctcattccgttgatgatcttagatattctctgcattaaatatcacctctaggctgagaaatccaccaaaaaatatttctagctcagcgttttcctccaaatcttcaatggaagatcataatgtgaactctgcatctccatgttaaagtttaatggacattcacacttagcatgtctcaaagaaatctcatgtaaaccatggccatcctgttctaccttaactttctgagtctatggaatgataatttcacatctcataaacttgactgatgtaagtgtcaagaaaagattgacattttgttaaaacttcgtagccaagtgtgtaacgcttaagcagactttcatatttcaaatctctatagcacgtgtaactcttttttcaagatgtgaaataatcattaggtcagtcatttgtaaatagtacggctgctgtgggctttttccagttcttcaccatccatttttataaaactcttattgttaaaaaaaaaaagttactcagaatttcataaagccaaacacctgatttcaggaacacttgagatgtaagaaaattttatagggacctccaatcactaattttcctattttttctctcaaagaaatgctgaagggaggaattcaggttgaatgaaaggaaatagtaacttacagccatatagagttataaagacttcttgtaaatgtgaacatatggtaaaatataaaaacatgtatttttgaaa

SEQ ID NO: 8 - DNA, 972 bp, Homo sapiens
  source 1..972: mol_type = genomic DNA
atggattcgaaataccagtgtgtgaagctgaatgatggtcacttcatgcctgtcctgggatttggcacctatgcgcctgcagaggttcctaaaagtaaagctctagaggccgtcaaattggcaatagaagccgggttccaccatattgattctgcacatgtttacaataatgaggagcaggttggactggccatccgaagcaagattgcagatggcagtgtgaagagagaagacatattctacacttcaaagctttggagcaattcccatcgaccagagttggtccgaccagccttggaaaggtcactgaaaaatcttcaattggactatgttgacctctatcttattcattttccagtgtctgtaaagccaggtgaggaagtgatcccaaaagatgaaaatggaaaaatactatttgacacagtggatctctgtgccacatgggaggccatggagaagtgtaaagatgcaggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaa

SEQ ID NO: 9 - protein, 323 aa, Homo sapiens
MDSKYQCVKLNDGHFMPVLGFGTYAPAEVPKSKALEAVKLAIEAGFHHIDSAHVYNNEEQVGLAIRSKIADGSVKREDIFYTSKLWSNSHRPELVRPALERSLKNLQLDYVDLYLIHFPVSVKPGEEVIPKDENGKILFDTVDLCATWEAMEKCKDAGLAKSIGVSNFNHRLLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 10 - DNA, 20 bp, synthetic construct
  source 1..20: mol_type = other DNA
gacaagcttcccgttctcag

SEQ ID NO: 11 - DNA, 20 bp, synthetic construct
  source 1..20: mol_type = other DNA
ggagtcaacggatttggtcg

SEQ ID NO: 12 - DNA, 27 bp, synthetic construct
  source 1..27: mol_type = other DNA
cctaaaagtaaagctttagaggccacc

SEQ ID NO: 13 - DNA, 30 bp, synthetic construct
  source 1..30: mol_type = other DNA
gaaaatgaataaggtagaggtcaacataat

SEQ ID NO: 14 - DNA, 26 bp, synthetic construct
  source 1..26: mol_type = other DNA
cctaaaagtaaagctctagaggccgt

SEQ ID NO: 15 - DNA, 29 bp, synthetic construct
  source 1..29: mol_type = other DNA
gaaaatgaataagatagaggtcaacatag

SEQ ID NO: 16 - DNA, 25 bp, synthetic construct
  source 1..25: mol_type = other DNA
gagaagtaaagctttggaggtcaca

SEQ ID NO: 17 - DNA, 29 bp, synthetic construct
  source 1..29: mol_type = other DNA
caacctgctcctcattattgtataaatga